SEQUENCE LISTING (19 sequences)

SEQ ID NO: 1 - DNA, 5540 bp, Homo sapiens
  human TEM8 nucleotide sequence
aattgcttccggggagttgcgagggagcgagggggaataaaggacccgcgaggaagggcccgcggatggcgcgtccctgagggtcgtggcgagttcgcggagcgtgggaaggagcggaccctgctctccccgggctgcgggccatggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaagaagatgatgatggtctgcctaagaaaaagtggccaacggtagacgcctcttattatggtgggagaggcgttggaggcattaaaagaatggaggttcgttggggagaaaagggctccacagaagaaggtgctaagttggaaaaggcaaagaatgcaagagtcaagatgccggagcaggaatatgaattccctgagccgcgaaatctcaacaacaatatgcgtcggccttcttccccccggaagtggtactctccaatcaagggaaaactcgatgccttgtgggtcctactgaggaaaggatatgatcgtgtgtctgtgatgcgtccacagccaggagacacggggcgctgcatcaacttcaccagggtcaagaacaaccagccagccaagtacccactcaacaacgcctaccacacctcctcgccgcctcctgcccccatctacactcccccacctcctgcgccccactgccctcccccgccccccagcgcccctacccctcccatcccgtccccaccttccacccttccccctcctccccaggctccacctcccaacagggcacctcctccctcccgccctcctccaaggccttctgtctagagcccaaagttcctgctctgggctctctcagaaacttcaggagatgttagaacaagtctttccagttagagaagaggagtggtgataaagcccactgaccttcacacattctaaaaattggttggcaatgccagtataccaacaatcatgatcagctgaaagaaacagatattttaaattgccagaaaacaaatgatgaggcaactacagtcagatttatagccagccatctatcacctctagaaggttccagagacagtgaaactgcaagatgctctcaacaggattatgtctcatggagaccagtaagaaaatcatttatctgaaggtgaaatgcagagttggataagaaatacattgctgggtttctaaaatgctgccttcctgcctctactccacctccatccctggactttggacccttggcctaggagcctaaggaccttcacccctgtgcaccacccaagaaagaggaaaactttgcctacaactttggaaatgctggggtccctggtgtggtaagaaactcaacatcagacgggtatgcagaaggatgttcttctgggatttgcaggtacataaaaaatgtatggcatcttttccttgcaaattcttccagtttccaagtgagaaggggagcaggtgtttactgatggaaaaggtatgttgctatgttgatgtgtaagtgaaatcagttgtgtgcaatagacaggggcgtattcatgggagcatcagccagtttctaaaacccacaggccatcagcagctagaggtggctggctttggccagacatggaccctaaatcaacagacaatggcattgtcgaagagcaacctgttaatgaatcatgttaaaaatcaaggtttggcttcagtttaaatcacttgaggtatgaagtttatcctgttttccagagataaacataagttgatcttcccaaaataccatcattaggacctatcacacaatatcactagttttttttgtttgtttgttttttgttttttttcttggtaaagccatgcaccacagacttctgggcagagctgagagacaatggtcctgacataataaggatctttgattaacccccataaggcatgtgtgtgtatacaaatatacttctctttggcttttcgacatagaacctcagctgttaaccaaggggaaatacatcagatctgcaacacagaaatgctctgcctgaaatttccaccatgcctaggactcaccccatttatccaggtctttctggatctgtttaatcaataagccctataatcacttgctaaacactgggcttcatcacccagggataaaaacagagatcattgtcttggacctcctgcatcagcctattcaaaattatctctctctctagctttccacaaatcctaaaattcctgtcccaagccacccaaattctcagatcttttctggaacaaggcagaatataaaataaatatacatttagtggcttgggctatggtctccaaagatccttcaaaaatacatcaagccagcttcattcactcactttacttagaacagagatataagggcctgggatgcatttattttatcaataccaatttttgtggccatggcagacattgctaatcaatcacagcactatttcctattaagcccactgatttcttcacaatccttctcaaattacaattccaaagagccgccactcaacagtcagatgaacccaacagtcagatgagagaaatgaaccctacttgctatctctatcttagaaagcaaaaacaaacaggagtttccagggagaatgggaaagccagggggcataaaaggtacagtcaggggaaaatagatctaggcagagtgccttagtcagggaccacgggcgctgaatctgcagtgccaacaccaaactgacacatctccaggtgtacctccaaccctagccttctcccacagctgcctacaacagagtctcccagccttctcagagagctaaaaccagaaatttccagactcatgaaagcaaccccccagcctctccccaaccctgccgcattgtctaatttttagaacactaggcttcttctttcatgtagttcctcataagcaggggccagaatatctcagccacctgcagtgacattgctggacccctgaaaaccattccataggagaatgggttccccaggctcacagtgtagagacattgagcccatcacaactgttttgactgctggcagtctaaaacagtccacccaccccatggcactgccgcgtgattcccgcggccattcagaagttcaagccgagatgctgacgttgctgagcaacgagatggtgagcatcagtgcaaatgcaccattcagcacatcagtcatatgcccagtgcagttacaagatgttgtttcggcaaagcattttgatggaatagggaactgcaaatgtatgatgattttgaaaaggctcagcaggatttgttcttaaaccgactcagtgtgtcatccccggttatttagaattacagttaagaaggagaaacttctataagactgtatgaacaaggtgatatcttcatagtgggctattacaggcaggaaaatgttttaactggtttacaaaatccatcaatacttgtgtcattccctgtaaaaggcaggagacatgtgattatgatcaggaaactgcacaaaattattgttttcagcccccgtgttattgtccttttgaactgttttttttttattaaagccaaatttgtgttgtatatattcgtattccatgtgttagatggaagcatttcctatccagtgtgaataaaaagaacagttgtagtaaattattataaagccgatgatatttcatggcaggttattctaccaagctgtgcttgttggtttttcccatgactgtattgcttttataaatgtacaaatagttactgaaatgacgagacccttgtttgcacagcattaataagaaccttgataagaaccatattctgttgacagccagctcacagtttcttgcctgaagcttggtgcaccctccagtgagacacaagatctctcttttaccaaagttgagaacagagctggtggattaattaatagtcttcgatatctggccatgggtaacctcattgtaactatcatcagaatgggcagagatgatcttgaagtgtcacatacactaaagtccaaacactatgtcagatgggggtaaaatccattaaagaacaggaaaaaataattataagatgataagcaaatgtttcagcccaatgtcaacccagttaaaaaaaaaattaatgctgtgtaaaatggttgaattagtttgcaaactatataaagacatatgcagtaaaaagtctgttaatgcacatcctgtgggaatggagtgttctaaccaattgccttttcttgttatctgagctctcctatattatcatactcagataaccaaattaaaagaattagaatatgatttttaatacacttaacattaaactcttctaactttcttctttctgtgataattcagaagatagttatggatcttcaatgcctctgagtcattgttataaaaaatcagttatcactataccatgctataggagactgggcaaaacctgtacaatgacaaccctggaagttgctttttttaaaaaaataataaatttcttaaatcaaaaaaaaaaaaaaaaaaaa

SEQ ID NO: 2 - protein, 564 aa, Homo sapiens
  human TEM8 protein sequence
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTGRCINFTRVKNNQPAKYPLNNAYHTSSPPPAPIYTPPPPAPHCPPPPPSAPTPPIPSPPSTLPPPPQAPPPNRAPPPSRPPPRPSV

SEQ ID NO: 3 - DNA, 5220 bp, Mus musculus
  mouse TEM8 nucleotide sequence
cggaactgcttcgactgcaaagcttcaagcgcagcctgggagcggcctggtggccctatcccggcagctccacacagcagaacgccctgggtccctgaaactcgaaacccgggctcagaaccagcggaaaccaaagcgaaatccttgaacttctctgaacaattgcttccgggcgtttgctgagagccgggggacctgaccggagcccaggccgcgtatggcgcgcccctgatgtcacacggacgccagcgaggccagcgctccggctgcagcatggaccgcgcggggcgcctgggtgcgggcctgcggggactctgcgtggctgcactcgtgctcgtgtgcgccggacacgggggccgccgcgaggatgggggaccagcttgctacggaggattcgacctctacttcatcctggacaagtcaggaagtgtgctgcaccactggaatgaaatctactacttcgtggagcagttggctcatagattcatcagcccacagctaaggatgtccttcattgtcttctctactcgagggacaactttaatgaaactaactgaggacagggaacagatccgacaaggcctagaagagctccagaaagttctgccaggaggagacacttacatgcacgaaggattcgagagggccagtgagcagatttactatgagaacagtcaaggatacaggacggcgagcgtcatcatcgcgttgacggatggggagctgcacgaggacctcttcttctactcagagagggaggctaaccgatcccgagaccttggtgcgattgtttactgcgttggcgtgaaggatttcaatgaaactcagttggctcggattgcagacagtaaggaccacgtgtttcctgtgaacgacggcttccaggctctccaaggcattatccactcaattttaaagaaatcctgcatcgaaattctggcggctgaaccatccaccatctgcgcgggagagtcctttcaagtggtcgtaagaggaaatggcttccgacatgcccgcaatgtggacagggtcctctgcagcttcaaaatcaatgactcagtcacgctcaatgagaagccctttgctgtggaagacacttatttgctgtgcccagcaccaatcttgaaagaagttggcatgaaagctgcactgcaggtcagcatgaacgacggcctgtccttcatctccagttctgtcatcatcaccaccacacactgttcagacggctccatcctggcgattgctctgctggtcctcttcctgctgctggccctggcgctgctctggtggttctggcccctctgctgcacagtgatcatcaaggaggtccctccaccccctgttgaggagagtgaggaagaagacgatgatggtttgccaaagaagaaatggcccacagtagatgcctcttattatggtggacgcggtgtgggaggcattaaaagaatggaggtccgctggggagaaaagggctccacagaagaaggggcgaagttagaaaaggcaaagaatgcacgagtcaagatgccagagcaagaatatgagttcccagaaccccgaaacctcaacaacaacatgcgccggccttcctcgcctcggaagtggtactcgcccatcaagggaaaactcgatgccttgtgggttctgctgagaaaaggatatgaccgagtgtctgtgatgaggccacagccaggagacacgggacgctgtatcaacttcaccagagtgaagaacagtcagccagccaagtatcccctgaacaacacctaccaccccagctccccacctcccgctcctatctacacacccccaccccctgctccccactgccctcccccagcccccagtgcccccactcctcccattccttccccaccatccactctcccccctcctcctcaggccccaccccctaacagggcacctcccccctcccgacctcctccaaggccttctgtctagaacccaaagtccgagctctgggctgcctgagcaactccagcaggaggcttctctgctgaaagaaagatctgcccagcctatgtggtgagtggcggctgatgtttgcacgatttaaaagcaagtcgtgatgggcagaacaaaatgggcattttgaactgcctgaagacagacaatgagacaataacagtcacattatagcctgtgacccctcacctctagaggaaggttcccgagatggccacattgccacagtgctctcagccagattatgtcccatgaagaccaggaagaaagtgacttccaagaatggaatgcagcattggataagaaacacctggctgagattctgacctcactgatttgactcttgattcttggactgggagccaggccatctccacccctggtaccacccagcaactctgaaaatgtgcagtgtccctagtatgcatcgaataggtatccaactgggatctgcaggttgccttataaagagcatatgctctattctctttcccgaacttcctggtttcccagtgatgagggaaggggaaaggtgttgccatgcttagaagttagaggacgtcagtgctcagcactgatggagaagcgttgatgggagtgtccagctcttacatctagaaatggctggcttcagcaggcacagttcctaaaccaacaagccttgtcattgtcaaaggcaacctactaatgattcaccttaaacatcaaggttgactgtggcataggtcagagctgatcacacagaaccttccccatgaaatcgcaaggttcctcatcttcaaatacccaggaccccagagatttctaaatccagctaagagacagtagtcctgacttggcaagaaaaccattcccagttgttttactctgaaacaggccgttgtatgtatggtatatctctccttggcctttcaacctgctcacaagtattaccagttatgaagcaaggagaaatacatccagtgtgtaatagaaaagctctgcccacaatccccatgtcactcctctacattattctgaagctgcttggtcagtgagccctttaacctcatgtagactctggacactgtcacccaatcatgaaaacagaggtcattgtcaaaggcagtgtatagcctgtacaaaaatgatgcttccttcctcagtttccacaggccccaaaattcctgtcttaggctcctaaacctctaaactttttcctggaacaaaagatataaaacgggcataagtttttatgttttgggctgtgatctccaaagatccttcaagaactcaagttagcctcattcttccagcttgtttagaacagaggcatccaggtgtcatgcactccatagacaccaatccttgttcccaaggcagacattattaatcaatctcagcactagttctcaatttaatccaattatatttttccacagtacttcacatctcttatgacctgttggtcatcagttagaattgagagagataaacactgtttgtaatccctaccttagaaagaaaagcagaggagaatgggggaaccaccagcataaaagttattatctggggaaaatcgacctgaaagaacgcccaagtccaagacctatggtgctgacaccaaagtaacactttcccaagtgtaccccagaccccactcttctccctgtggccaccactccctgcttttcaggagttgtgaaaaagatctccttcacccttactgtgcccccatattagaacaaggcttgtttagtgtagtccttgttaaacaggtgccagaatgtctcagccacctgagatgacattgctgggccccagaaaaccattccaaggagaatgggctccccaggctcagagcatgcaactatgagcccatggcaactgttttgactgctggcagtacaaaacgggccaccccacattacagctgcaggatttgtgcagccataagaaagtatgaaccaagatgctggtgttgctgttcaacaagcatgggcttcggggaaggcagcagactccgagagcaggccttgtgcagtgtcccaaggggctgtggtgaagtgtctgaggaaaaatgaatgctgatacatggtgattctgagaagaatttgcaaggtttgaccttagaatttatggaatgtcttccctggtcattcagaattatggctagaagtttctagaaaccgtcaaggttaatacctttcagagtaggtgattacaggcaggaagagctttgatgtggtttacaaagcccatcagttctgtgtcattccctgtaagcaacaggagatggtggttgtgattagcaaactgcatgtgttatttgtttgactccttgttattgtccttacggaggattttttttatataagccaaattttgttgtatatattcatattccacgtgacagatggaagcacgtcctatcagtgtgaataaaaagaacagttgtagtaaattattaaagccagtgatttcatggcaggttaccctaccaagctgtgcttgttgatctcccatgaccatactgcttttacaatgtacaaatagttcctaggtgacgagaccctcctttacataatgccgatgacagccttgctgggaactgcggtccttctgctgtgacagccagctcgaaaacaggtcctgcctggagcttgccacacactttagggagacataagagctgtctttccccagcgtcagggacaaagctaccataaagaagtggaaaagtcttggctctccagcctgggacagaggtctctctggaaccccaaggaagagcagaaatgatccttgcctgccactgcacacaatgtgatggtggaaaatccatcaaggaataattgtgagataatgaccgacagttcaggcgcaaagggaattcatgctgtgtaaagtgggtggaattcgtttgcaagctatgcaaagcctgatcttactcaccaggaggatggaaagggtttttttagttatctgagctcagctgagttatcacgcttggagaaccgatttaaaggaattagaatatgatttctgaatacacataacattaaactcttctctttttctatggtaatttagttatggacgttcagcgtctctgagttattgttataaaagacttgtcatcaccgcactgtgctgtaggagactgggctgaacctgtacaatggtataccctggaagttgcttttttaaaaaaaaataataataaacacctaaaatcaaaaaaaaaaaaaaaaa

SEQ ID NO: 4 - protein, 562 aa, Mus musculus
  mouse TEM8 protein sequence
MDRAGRLGAGLRGLCVAALVLVCAGHGGRREDGGPACYGGFDLYFILDKSGSVLHHWNEIYYFVEQLAHRFISPQLRMSFIVFSTRGTTLMKLTEDREQIRQGLEELQKVLPGGDTYMHEGFERASEQIYYENSQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFAVEDTYLLCPAPILKEVGMKAALQVSMNDGLSFISSSVIITTTHCSDGSILAIALLVLFLLLALALLWWFWPLCCTVIIKEVPPPPVEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTGRCINFTRVKNSQPAKYPLNNTYHPSSPPPAPIYTPPPPAPHCPPPAPSAPTPPIPSPPSTLPPPPQAPPPNRAPPPSRPPPRPSV

SEQ ID NO: 5 - DNA, 29 bp, Artificial Sequence
  forward primer for extracellular domain of rat HER2/neu
cgaagcttaccatggagctggcggcctgg

SEQ ID NO: 6 - DNA, 26 bp, Artificial Sequence
  reverse primer for extracellular domain of rat HER2/neu
cggaattcttatgtcaccgggctggc

SEQ ID NO: 7 - DNA, 25 bp, Artificial Sequence
  forward primer for amino acids 13-357 of the extracellular domain of mouse TEM8
ggactctgcgtggctgcactcgtgc

SEQ ID NO: 8 - DNA, 25 bp, Artificial Sequence
  reverse primer for amino acids 13-357 of the extracellular domain of mouse TEM8
agagcagcgccagggccagcagcag

SEQ ID NO: 9 - DNA, 36 bp, Artificial Sequence
  forward primer for amino acids 27-289 of the extracellular domain of mouse TEM8
gggggtaccgcaatgggccgccgcgaggatggggga

SEQ ID NO: 10 - DNA, 34 bp, Artificial Sequence
  reverse primer for amino acids 27-289 of the extracellular domain of mouse TEM8
ggtggaattcctagcacagcaaataagtgtcttc

SEQ ID NO: 11 - DNA, 21 bp, Artificial Sequence
  forward primer for intracellular domain of mouse TEM8
gaagacgatgatggtttgcca

SEQ ID NO: 12 - DNA, 21 bp, Artificial Sequence
  reverse primer for intracellular domain of mouse TEM8
gtggtaggtgttgttcagggg

SEQ ID NO: 13 - DNA, 55 bp, Artificial Sequence
  forward primer for mouse TEM8 DNA
ggggacaagtttgtacaaaaaagcaggcttgatgggccgccgcgaggatggggga

SEQ ID NO: 14 - DNA, 51 bp, Artificial Sequence
  reverse primer for mouse TEM8 DNA
ggggaccactttgtacaagaaagctgggtcgcacagcaaataagtgtcttc

SEQ ID NO: 15 - protein, 8 aa, Artificial Sequence
  mouse TEM8 peptide
ACYGGFDL

SEQ ID NO: 16 - protein, 9 aa, Artificial Sequence
  mouse TEM8 peptide
SVLHHWNEI

SEQ ID NO: 17 - protein, 8 aa, Artificial Sequence
  mouse TEM8 peptide
IYYFVEQL

SEQ ID NO: 18 - protein, 8 aa, Artificial Sequence
  mouse TEM8 peptide
NSQGYRTA

SEQ ID NO: 19 - protein, 8 aa, Artificial Sequence
  control ovalbumin peptide
SIINFEKL

(19) United States
(12) Patent Application Publication
(10) Pub. No.: US 2010/0310603 A1
(43) Pub. Date: Dec. 9, 2010

(54) TEM8 as an Adjuvant and Uses Thereof

(76) Inventors: Polly Gregor, New York, NY (US); Alan Houghton, New York, NY (US)

Correspondence Address: Adler & Associates, 8011 Candle Lane, Houston, TX, 77071, US

(21) Appl. No.: 12/225,508

(22) PCT Filed: Mar. 23, 2007
(86) PCT No.: PCT/US2007/007273

§ 371 Date: Nov. 23, 2009

Related U.S. Application Data
(60) Provisional application No. 60/785,393, filed on Mar. 23, 2006.

(51) Int. Cl. A61K 39/21; A61K 39/00; A61K 39/02; A61K 39/07; A61K 39/015; A61K 38/02; A61P 37/04; A61P 35/00; A61P 35/02; A61P 33/06; A61P 31/04; A61P 31/18

(52) U.S. Cl. 424/208.1; 424/184.1; 424/277.1; 424/234.1; 424/246.1; 424/272.1; 514/13.3

(57) ABSTRACT

The present invention discloses a composition comprising an immunogenic sequence or a fragment thereof and TEM8 or a fragment thereof, where the TEM8 or the fragment functions as an adjuvant and enhances the eilicitation of immune responses mediated by the immunogenic sequence or the fragment thereof. Also disclosed herein is the use of such compositions in the treatment of cancer or pathogen associated diseases.

### CROSS-REFERENCE TO RELATED APPLICATIONS

This U.S. national stage application is filed under 35 U.S.C. §363 and claims benefit of priority under 35 U.S.C. §365 of international application PCT/US2007/007273, filed Mar. 23, 2007, now abandoned, which claims benefit of priority under 35 U.S.C. §119(e) of provisional U.S. Ser. No. 60/785,393, filed Mar. 23, 2006, now abandoned.

### BACKGROUND OF THE INVENTION

1. Field of the Invention

The present invention relates generally to the field of immunology. In general, the present invention discloses compositions comprising TEM8, preferably human TEM8, as an active ingredient and administering such compositions for enhancing the immune response to vaccination in animals, including humans. More specifically, the present invention relates to the use of TEM8 as an adjuvant with immunogenic sequences that can function as a vaccine.

2. Description of the Related Art

The use of vaccines to prevent diseases in humans and animals is a common practice, and considerable effort has been, and is being made to extend this practice to cover a more extensive array of diseases to which these patients are subject. For example, the use of rabies vaccine in animals is now commonplace, and efforts are being made to obtain suitable vaccines to immunize animals against other diseases. More recently, the use of vaccines to treat cancer is being intensely studied and have started to be used in humans.

However, one problem that is frequently encountered in the course of active immunization is that the antigens used in the vaccine are not sufficiently immunogenic to induce a strong cell-mediated immunity. Notorious among such weak animal vaccines are those constituted from inactivated Haemophilus pleuropneumoniae (Hpp) (which is associated with respiratory disease in pigs).

In order to obtain a stronger humoral and/or cellular response, it is common to administer the vaccine in a formulation containing an adjuvant (immunopotentiator), a material that enhances the immune response of the patient to the vaccine. The most commonly used adjuvants for vaccines are oil preparations and alum. The mechanisms by which such adjuvants function are not understood, and whether or not a particular adjuvant preparation will be sufficiently effective in a given instance is not predictable.

TEM8 is a member of a recently discovered family of proteins associated with tumor-specific angiogenesis. TEM8 RNA was originally isolated from a human colorectal tumor and its expression was reported to be restricted to tumor vasculature (Carson-Walter et al., 2001; Nanda et al., 2004). Also, TEM8 has been found to be expressed in vascular endothelial cells and tumor stroma.

It is known that TEM8 behaves in some manner in vivo able to enhance and increase a successful immune response in cancer and it was proposed that this response is related to TEM8 expression in tumor vasculature, creating a synergistic immune response when given in conjunction with a cancer specific antigen. However, the absence of an immune response specific to TEM8 when TEM8 is administered as a DNA vaccine indicates that TEM8 must be acting in a different way (Felicetti et al., 2007). Thus, the mechanism by which TEM8 mediates or potentiates/enhances an immune response is not known in the art.

Taken together, there is a need for additional effective adjuvant preparations suitable for potentiating vaccines for animals including humans and other mammals. Additionally, there is a lack of understanding of the mechanism by which TEM8 mediates or potentiates/enhances an immune response. The present invention fulfills these long-standing needs and desires in the art.

### SUMMARY OF THE INVENTION

In one embodiment of the present invention, there is a composition comprising an immunogenic sequence or a fragment thereof, a TEM8 sequence or a fragment thereof, a pharmaceutically acceptable carrier or a combination thereof.

In another related embodiment of the present invention, there is a method of eliciting an enhanced immune response in a subject. This method comprises the step of administering an immunologically effective amount of the composition described supra to the subject.

In yet another embodiment of the present invention, there is a composition comprising an immunogenic sequence or a fragment thereof, an amino acid sequence that is at least 90% homologous to an amino acid sequence of TEM8 or a fragment thereof, a pharmaceutically acceptable carrier or a combination thereof.

In another related embodiment of the present invention, there is a method of eliciting an enhanced immune response in a subject. Such a method comprises the step of administering an immunologically effective amount of the composition described supra to the subject.

In still yet another embodiment of the present invention, there is a composition comprising an immunogenic sequence or a fragment thereof, an amino acid sequence that is at least 80% homologous to an amino acid sequence of TEM8 or a fragment thereof, a pharmaceutically acceptable carrier or a combination thereof.

In another related embodiment of the present invention, there is a method of eliciting an enhanced immune response in a subject. Such a method comprises the step of administering an immunologically effective amount of the composition described supra to the subject.

In another embodiment of the present invention, there is a composition comprising a TEM8 or a fragment thereof and a pharmaceutically acceptable carrier.

In another related embodiment of the present invention, there is a method of increasing the ability of an immunogenic composition to elicit an immune response in a subject. Such a method comprises the step of administering the composition comprising TEM8 or the fragment thereof and a pharmaceutically acceptable carrier to the subject.

In yet another embodiment of the present invention, there is a composition comprising an amino acid sequence that is at least 90% homologous to an amino acid sequence of TEM8 or a fragment thereof and a pharmaceutically acceptable carrier. In another related embodiment of the present invention, there is a method of increasing the ability of an immunogenic composition to elicit an immune response in a subject. Such a method comprises the step of administering the composition comprising the sequence homologous to TEM8 or the fragment thereof and a pharmaceutically acceptable carrier described supra to the subject.

In still yet another embodiment of the present invention, there is a composition comprising an amino acid sequence that is at least 80% homologous to an amino acid sequence of TEM8 or a fragment thereof and a pharmaceutically acceptable carrier. In another related embodiment of the present invention, there is a method of increasing the ability of an immunogenic composition to elicit an immune response in a subject. Such a method comprises the step of administering the composition comprising the sequence homologous to TEM8 or the fragment thereof and a pharmaceutically acceptable carrier described supra to the subject.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1 shows that tumor-specific T cells are enriched by in vivo expansion in tumors. Mice immunized 3 times with an ovalbumin DNA vaccine were challenged with either B16 tumor (ova-) or MO4 tumor (B16 expressing ova). Six days later, draining lymph nodes (left panel) and matrigel plugs (right panel) were recovered, and ova-specific CD8 T cells were measured by IFN-g ICCA.

FIG. 2 shows that tumor-specific T cells are enriched in tumors. Mice immunized 3× with HER2/neu DNA vaccine were challenged with 233-VSAG-1 tumor. Ten days later, neu-specific CD8 T cells were measured by ICCA from spleen and matrigel plugs.

FIG. 3 shows that TEM8 injection increases the number of neu-specific T cells. Mice (5/group) received 3 injections of TEM8, rat HER2/neu, or combinations as indicated. Five days after the last injection, 233-VSAG-1 tumor was implanted in matrigel in the flank, and 10 days later, plugs were removed and TILS assayed by ICCA.

FIGS. 4A-4F show that TEM8 injection increases the number of antigen-specific T cells in draining lymph nodes of immunized mice. In FIG. 4A, mice (3/group) received 5 injections of TEM8, rat HER2/neu, or combinations as indicated. 5 days after the last injection, CD8 T cells were isolated from draining lymph nodes and assayed by IFN-g ELISpot. In FIG. 4B, mice (3/group) were immunized with hgp75 alone or in combination with TEM8. Five days after the last injection, CD8 T cells were isolated from draining lymph nodes and assayed by IFN-g ELISpot for recognition of three immunodominant gp75 peptides 455, 451 and 522. TEM8 increased the response to peptides 455 (and to a lesser extent 481 and 522) in mice immunized with hgp75. In FIG. 4C, mice (3/group) were vaccinated three times with ova DNA+/−TEM8 or pING, or once with SINFEKL (SEQ ID NO: 19) peptide in Titermax. On day 5, CD8 T cells were isolated from draining LNs and assayed by IFN-γ ELISPOT. TEM8 increased the number of ova-specific CD8+ T cells approximately two-fold, showing the ability of TEM8 to increase CD8+ T cell responses to both foreign and self antigens. FIGS. 4D and 4E show that CD8+ T cells contribute to TEM8 immunity. In FIG. 4D, FVB/neuNT transgenic mice were immunized three times bi-weekly by intramuscular injection with 100 μg pcDNA3, TEM8 or Her2/neu (left panel) and challenged with 233-VSAG1 tumor cells. Tumor-free survival is reported using Kaplan-Meier analysis. An additional group of mice immunized with HER2/neu was depleted of CD8 T cells by injection with MAb (53-6.7.2) for 4 weeks beginning 3 days prior to tumor challenge. For clarity, tumor-free survival data for additional groups of mice from the same experiment, TEM8+HER2/neu and TEM8+HER2/neu immunized mice depleted of CD8 T cells are represented in the right panel. In FIG. 4E, C57BL/6 were immunized five times weekly by particle bombardment with 4 μg DNA encoding TEM8, hTYRP1/hgp75 or the two vaccines in combination. In one group, mice were depleted of CD8+ T cells by injection with MAb (53-6.7.2) for 4 weeks beginning 3 days prior to tumor challenge. Tumor-free survival following intradermal challenge with B16F10 melanoma is reported using Kaplan-Meier analysis. FIG. 4F shows that TEM8 adjuvant effect increases CD4+ T cell activation and the increase in CD8+ T cells requires CD4+ T cells. FVB mice received 3 injections of rat HER2/neu+/−TEM8 or pING. In some groups, mice were depleted of CD4+ T cells throughout immunization. Five days after the last vaccine, purified CD8+(left) or CD4+(right) T cells were assayed by IFN-γ ELISPOT.

FIGS. 5A-5B shows that mutations in TEM8 injection decrease its adjuvant effect. In FIG. 5A, mice received 3 injections of rat HER2/neu (left) or hgp75 (right), +/−pING, TEM8 or mutated TEM8 (Opt2TEM8) DNA. At day 5, CD8T cells from lymph nodes were assayed by ELISPOT. In FIG. 5B, COS7 cells were transfected with (FLAG-tagged) Opt-2 or TEM8 DNA. After 20 hours, cells were pulsed with Muconmycin A for 1 hour, washed 4 times with PBS and incubated in complete media for an additional 0-24 hours as indicated.

FIG. 6 shows that individual cells in a mouse prostate tumor expresses TEM8. Two days following castration, a spontaneous tumor arising in a Pten−/− p53−/− mouse was stained with a commercial antibody specific for TEM8 (ABR, Santa Cruz).

FIGS. 7A-7O show that TEM8 is expressed in cells within breast and melanoma tumors, human prostate tumors and in monocytes/macrophages and dendritic cells. FIGS. 7A-7I show expression of TEM8 in mouse breast and melanoma tumors. In situ hybridization detects TEM8 RNA in the stroma of transplantable tumors B16F10 (FIGS. 7A-7D) and 233-VSGA1 (FIGS. 7E-7H). Dark field images are in (FIGS. 7A, 7C, 7E and 7G) and bright field are in (FIGS. 7B, 7D, 7F and 7H). A Western blot developed with the ATR/TEM8-specific Ab N19 detects TEM8 protein in the same tumor types (FIG. 7I). Lysates (30 μg total protein) were prepared from breast and melanoma cell lines. Lane 1, 233-VSGA1 breast tumor; lane 2, 233-VSGA1 in vitro culture, lane 3, B16F10 tumor; lane 4, B16F10 in vitro culture; lane 5, FVB normal kidney. Asterix indicates a 80-kDa band in 233-VSGA1 and B16F10 tumor samples and normal kidney but not in cultured 233-VSGA1 or B16F10 cells (arrows indicate areas of hybridization to the TEM8 antisense probe). FIGS. 7J-7N show that antigen presenting cells (APC) express TEM8 in vitro and in vivo and FIG. 70 shows that TEM8 expression increases after LPS stimulation. In FIG. 7J, immunohistochemical analysis shows expression of TEM8 (left panel) and CD68 (right panel) in paraffin sections of human prostate cancer. In FIGS. 7K-7L, flow cytometry shows TEM8 expression (red line) in APCs within the tumor (FIG. 7K) and lymph nodes (FIG. 7L) of naïve and B 16 bearing mice (TDLN). TEM8 expression in bone marrow derived dendritic cells (DCs) as analyzed by Western blot (FIG. 7M) after different days of culture and flow cytometry (FIG. 7N) at day 15. In FIG. 70, mouse bone marrow was cultured in GM-CSF for 10 days to prepare DCs or in G-CSF for 5 days to prepare macrophages (top). Day 11 DCs were treated with LPS for 3, 6, 9 or 12 hours (bottom). Day 5 macrophages were treated with LPS for 24 hours. In both cases, TNFα and TEM8 RNA was measured by quantitative RTPCR analysis.

FIGS. 8A-8C show that co-injection of HER2/neu and TEM8 DNA confers tumor protection. In FIG. 8A, the FVB/NT transgenic mice were immunized bi-weekly by intramuscular injection with 100 μg DNA encoding TEM8, HER2/neu or the two vaccines in combination. A control group of mice received an empty vector pcDNA3. Tumor-free survival after challenge with 233-VSGA1 tumor is shown using Kaplan-Meier analysis. In FIG. 8B, FVB/N parental, non-transgenic mice were immunized five times weekly by particle bombardment with 4 μg DNA encoding TEM8, HER2/neu or the two vaccines in combination. In the TEM8+HER2/neu group, mice were immunized with TEM8 vaccine on day 1 and with HER2/neu on day+3. A control group of mice received no immunizations (naïve). Tumor-free survival after challenge with 233-VSGA1 tumor is shown over time using Kaplan-Meier analysis. In FIG. 8C, growth curves are plotted for individual mice used in the experiment described in FIG. 8B. Four out of the 15 mice immunized with TEM8+ HER2/neu showed tumor rejection after initial tumor growth (dashed line).

FIGS. 9A-9B show effect of administering DNA encoding TEM8, hTYRP1/hgp75, HER2.neu or combinations thereof. FIG. 9A shows that combined TEM8 and bTYRP1/hgp75 DNA vaccine increased protection against challenge with B16F10 melanoma. C57B16 mice were immunized five times weekly by particle bombardment with 4 μg DNA encoding TEM8, hTYRP1/hgp75 or the two vaccines in combination. A control group of mice received no immunization (naïve). Tumor-free survival after the intradermal challenge with B16F10 melanoma is shown using Kaplan Meier analysis. FIG. 9B shows that antibody to TEM8 were not detected following immunization. Mice were immunized five times weekly by particle bombardment with the indicated vaccines. Sera were collected 5 days after the last vaccine, pooled and analyzed for Antibody specific for recombinant TEM8 using Western blot analysis. H140 polyclonal antibody was used as a positive control.

FIG. 10 shows that TEM8 administration increases dendritic cell migration from the skin to the lymph node. Mice received a single administration of either TEM8 DNA or empty vector (mock) gene gun, At the indicated days, mice were sensitized with FITC to track the DC migration. 24 hours later, the FITC content in the lymph node DCs was measured by flow cytometry. Top: The CD11cint MHC class II high DC population among CD3-CD19-DAPI-cells (left) are analyzed for FITC content (right). Bottom: The percentage of CD11c+F1TC+ cells in the lymph node is reported. Each symbol represents one mouse.

FIG. 11 shows that TEM8 RNA is reduced in COS 7 cells transfected with selected TEM8 siRNA. COST cells were transfected with siRNA #1, #2, #3 or #AM. After 24 hours, TEM8 DNA was quantified by qRT-PCR, normalized to untreated cells and 18S RNA.

FIGS. 12A-12B show TEM81oxP knock-in targeting strategy. FIG. 12A shows the TEM8 gene which is 200 Kb long and contains 22 exons. The targeting vector contains a Neomycin cassette (NEO) flanked by two flippase recombination sites (Frt), exons 2 and 3 (Exons), two Lox P sites and 5′ (5-arm) and 3′ (3-arm) DNA sequences to allow homologous recombination. FIG. 12B shows results of southern blot analysis where probes X and y were tested on EcoRV digested C57B6 DNA.

### DETAILED DESCRIPTION OF THE INVENTION

### Definitions

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “adjuvant” has its conventional meaning, i.e., the ability to enhance the immune response to a particular antigen. Such ability is manifested by a significant increase in immune-mediated protection. An enhancement of humoral immunity is typically manifested by a significant increase (usually >10%) in the titer of antibody raised to the antigen. Similarly, enhancement of cellular immunity is typically manifested by a significant increase (usually>10%) in the number of responding CD8+ or CD4+ T cells.

As used herein, “TEM8” refers to a polypeptide obtained from tissue cultures or by recombinant techniques exhibiting the spectrum of activities characterizing this protein. The word includes not only human TEM8 (hTEM8), but also other mammalian TEM8 such as, e.g., dog, cat, mouse, rat, rabbit, primate, horse, pig and bovine TEM8. The nucleotide sequence and amino acid of native human TEM8 is shown in SEQ ID Nos 1 and 2. This primary amino acid and nucleotide sequence may be obtained as the native protein/DNA from natural sources or may be recombinantly derived.

As used herein, the term “DNA vaccines” are defined as purified preparations of plasmid DNA designed to contain one or more genes or fragments of those genes as well as regulatory genetic elements to enable production in a bacterial host system. Typically, these plasmids possess DNA sequences necessary for selection and replication in bacteria. In addition, they contain eukaryotic promoters and enhancers as well as transcription termination/polyadenylation sequences to promote gene expression in vaccine recipients, and may contain immunomodulatory elements.

It is an object of the present invention to provide a preparation comprising nucleic acid molecules encoding a tumor-associated or a pathogen associated antigen(s), or their immunogenic or their protein product, full length or fragments or derivatives, and a TEM8 gene or protein product, full length or fragment, to be used simultaneously, separately or sequentially for preventive or therapeutic treatment of cancer or of an infectious disease.

The present invention provides vaccine compositions and methods of adding an adjuvant to vaccines intended to provide a protective cell-mediated immune response in vaccinated host mammals against certain pathogens or against certain cancers using as an adjuvant TEM8. Most desirably, the invention is directed to vaccines which rely on enhancing the vaccinated host's cell-mediated immunity, i.e., the elicitation of helper and cytotoxic T lymphocytes (CTLs) and activated phagocytes, to provide protection against infection by the selected pathogen or against the tumor progression, in the case of tumor immunity.

The present invention demonstrates that injection of TEM8 in an animal increased dendritic cell migration and T cell responses to vaccines given in concert. TEM8 injection increased tumor immunity in a CD8 T cell-dependant fashion, despite the apparent lack of a specific immune response against TEM8. The present invention demonstrates that the increased tumor immunity observed subsequent to TEM8 injection was due to an adjuvant effect of the TEM8 DNA, and that TEM8 DNA injection increased dendritic cell migration from the skin to the draining lymph nodes and also increased CD4+ and CD8+ T cell responses.

Contrary to previous studies on TEM8 expression, where its localization was reported to be restricted to tumor vasculature, TEM8 is expressed in activated antigen presenting cells (APC) in the vicinity of the tumors in mouse and man. The present invention demonstrates that TEM8 is not detected in freshly isolated mouse bone marrow, but is expressed in dendritic cells after in vitro culture in GM-CSF and the expression is further increased by stimulation an maturation of these cells with LPS. TEM8 is also expressed in LPS-activated macrophages and in CD11b+macrophages present in mouse breast tumors and TEM8 cells co-localize with CD68+ cells (macrophages) in human breast and prostate tumors.

From TEM8's structure, expression site and involvement in cell migration in vascular endothelial cells, and that TEM8 increases the number or mobility of antigen presenting cells in the draining lymph nodes (dLN), it is concluded that TEM8 increases antigen presentation. The increase in CD8 T cells might reflect a change in the cytokine milieu or promote maturation of macrophages into M1 cells instead of M2.

TEM8/ATR is expressed on dendritic cells and macrophages, and anthrax infection causes a profound decrease in the functional response to infection. TEM8 is very conserved in evolution, and must have an important biological function that is unrelated to its role as a receptor for anthrax. In addition to their role as antigen presenting cells and phagocytes, macrophages play a role in breast development, breast bud modeling and placental development. TEM8+ cells in the normal breast and in the placenta are likely to be macrophages. TEM8 interacts with collagen and it is important in cell migration, and has been implicated in tumor angiogenesis.

The data herein provides support for a role for TEM8 in antigen presentation. An intact extracellular domain of TEM8 is needed to increase T cell responses, and from these results TEM8 protein may have an active role in increasing antigen presentation and that mutations in the vWf domain are needed for that activity. These same mutations may be tested for their ability to inhibit cell migration and anthrax binding, to dissect these biological activities.

There are two scenarios for the mechanism by which TEM8 influences antigen presentation. It is likely that TEM8 on antigen presenting cells may interact with another protein to increase motility, phagocytosis or maturation. In this case, injection of TEM8 DNA may increase expression of TEM8 in antigen presenting cells and amplify this effect. Alternately, if TEM8 acts normally to repress antigen presenting cell activation, TEM8 DNA may provide a soluble TEM8 that may interfere with this interaction, much as sTEM8 interferes with anthrax binding. The present data favors the first scenario, as a longer TEM8 construct, containing the transmembrane domain, was still active as an adjuvant. Clearly, now that TEM8 has been described as a general immune adjuvant, it will be useful both in the field of tumor immunology and in the field of pathogen vaccines. In other words, the immunogenic composition described herein will be useful for both tumor immunotherapy and attenuation of, or prophylatic treatment for infectious diseases. Most importantly, these compositions will be not only be useful in times of epidemics but also be useful for soldiers and civilians alike when they are exposed or suspected to be exposed to biological weapons. These compositions may also be useful in companion animals. The present invention also contemplates designing small molecule inhibitors of TEM8 and determining if they substitute for TEM8 DNA injection.

In one embodiment of the present invention, TEM8 may be administered as full length TEM8 or as a fragment of TEM8. One possible fragment is the one comprising the amino acids 28-278 of TEM8, or the corresponding nucleic acid sequence encoding amino acids from 28-278 of the mouse TEM8 sequence of SEQ ID Nos 3 and 4. Additionally, a person having ordinary skill would readily recognize that the TEM8 amino acid sequence or nucleic acid sequence may be manipulated to produce a useful TEM8 that is not 100% identical to either the protein or the nucleic acid sequence of SEQ ID Nos 3 and 4. For example, a person having ordinary skill would find useful a protein or nucleic acid that is 80% or 90% homologous to the sequence of SEQ ID Nos 3 and 4. Substantial identity of amino acid sequences means the sequences are identical or differ by one or more amino acid alterations (additions, substitutions) which do not cause an adverse functional dissimilarity between the synthetic protein and native human TEM8.

The present invention is directed to a composition comprising an immunogenic sequence or a fragment thereof, a TEM8 or a fragment thereof, a pharmaceutically acceptable carrier or a combination thereof. The immunogenic sequence or the fragment thereof and the TEM8 or the fragment thereof in general, may be a recombinant protein or a peptide. The recombinant protein or the peptide may comprise of modified amino acids, unmodified amino acids or both. The TEM8 or the fragment thereof in the composition may have an amino acid sequence shown in SEQ ID NO: 2 or SEQ ID NO: 4.

Alternatively, a nucleotide sequence encoding the immunogenic sequence or the fragment thereof and a nucleotide sequence encoding the TEM8 or the fragment thereof may be inserted in a vector. The vector in such a case may be a plasmid, a viral vector or a bacterial vector. Additionally, the nucleotide sequence encoding the TEM8 or the fragment thereof may comprise at least 60 nucleotides. Furthermore, the nucleotide sequence encoding TEM8 or the fragment thereof in the composition may have the sequence shown in SEQ ID NOS: 1 or 3. The immunogenic sequence of the fragment thereof may be an immunogenic sequence or a fragment thereof of a tumor associated antigen or a pathogen-associated antigen. Examples of the tumor associated antigen may include but are not limited to Her2/neu, gp75, Her2/NEU, gp75/TYRP-1, PSMA, TRY P-2, NY-ESO-1, MAGE-1, MAGE-3, CEA, PSA, tyrosinase, mutant p53, mutant p21, mutant cdk4, mutant L9, BCR-ABL or E6/E7 of HPV16 and those of the pathogen-associated antigen may include but are not limited to bacterial lipopolysaccharide, anthrax lethal factor, anthrax edema factor, HIV gp120 or malaria antigens such as CSP, TRAP/SSP2, LSA1, MSP1 and AMA1.

The present invention is also directed to a method of eliciting an immune response in a subject, comprising the step of administering an immunologically effective amount of the composition described supra to the subject. The TEM8 or the fragment thereof in the composition may increase the ability of the immunogenic sequence or the fragment thereof in the composition to elicit an antibody response, a CD4 T cell response, a CD8 T cell response or a combination thereof against an antigen associated with a tumor or against a pathogen in the subject. The representative examples of the antigen associated with a cancer and a pathogen are the same as described supra. Additionally, the subject benefiting from such a method may include but is not limited to one with breast cancer, prostate cancer, melanoma, colon cancer, chronic myeloid leukemia or cervical cancer or has been exposed or suspected of being exposed to malaria, anthrax, HIV or biological weapons.

The present invention is also directed to a composition comprising an immunogenic sequence or a fragment thereof, an amino acid sequence that is at least 90% homologous to an amino acid sequence of TEM8 or a fragment thereof, a pharmaceutically acceptable carrier or a combination thereof. The amino acid sequence of TEM8 or the fragment thereof may have a sequence shown in SEQ ID NO: 2 or SEQ ID NO:4. Additionally, the immunogenic sequence or the fragment thereof and the sequence homologous to TEM8 or the fragment thereof may be a recombinant protein or a peptide. The recombinant protein or the peptide may comprise of modified amino acids, unmodified amino acids or both.

Alternatively, a nucleotide sequence encoding the immunogenic sequence or the fragment thereof and a nucleotide sequence encoding the sequence homologous to TEM8 or the fragment thereof may be inserted in a vector. The vector in such a case may be a plasmid, a viral vector or a bacterial vector. Additionally, the nucleotide sequence encoding the sequence homologous to TEM8 or the fragment thereof may comprise at least 60 nucleotides. Furthermore, the nucleotide sequence encoding TEM8 or the fragment thereof in the composition may have a sequence of SEQ ID NOS: 1 or 3. Additionally, examples of the types of immunogenic sequences or the fragments thereof are the same as described supra.

The present invention is further directed to a method of eliciting an immune response in a subject, comprising the step of administering an immunologically effective amount of the composition described supra to the subject. The sequence homologous to TEM8 or the fragment thereof in the composition may increase the ability of the immunogenic sequence or the fragment thereof in the composition to elicit an antibody response, a CD4 T cell response, a CD8 T cell response or a combination thereof against an antigen associated with a tumor or against a pathogen in the subject. The representative examples of the antigen associated with a cancer and a pathogen and the type of subject benefiting from such a method are the same as described supra.

The present invention is also directed to a composition comprising an immunogenic sequence or a fragment thereof, an amino acid sequence that is at least 80% homologous to an amino acid sequence of TEM8 or a fragment thereof, a pharmaceutically acceptable carrier or a combination thereof. The amino acid sequence of TEM8 or the fragment thereof may have a sequence shown in SEQ ID NO: 2 or SEQ ID NO:4. Additionally, the immunogenic sequence or the fragment thereof and the sequence homologous to TEM8 or the fragment thereof may be a recombinant protein or a peptide. The recombinant protein or the peptide may comprise of modified amino acids, unmodified amino acids or both.

Alternatively, a nucleotide sequence encoding the immunogenic sequence or the fragment thereof and a nucleotide sequence encoding the sequence homologous to TEM8 or the fragment thereof may be inserted in a vector. The vector in such a case may be a plasmid, a viral vector or a bacterial vector. Additionally, the nucleotide sequence encoding the sequence homologous to TEM8 or the fragment thereof may comprise at least 60 nucleotides. Furthermore, the nucleotide sequence encoding TEM8 or the fragment thereof in the composition may have a sequence shown in SEQ ID NOS: 1 or 3. Additionally, examples of the types of immunogenic sequences or the fragments thereof are the same as described supra.

The present invention is further directed to a method of eliciting an immune response in a subject, comprising the step of administering an immunologically effective amount of the composition described supra to the subject. The sequence homologous to TEM8 or the fragment thereof in the composition may increase the ability of the immunogenic sequence or the fragment thereof in the composition to elicit an antibody response, a CD4 T cell response, a CD8 T cell response or a combination thereof against an antigen associated with a tumor or against a pathogen in the subject. The representative examples of the antigen associated with a cancer and a pathogen and the type of subject benefiting from such a method are the same as described supra.

The present invention is also directed to a composition comprising a TEM8 or a fragment thereof and a pharmaceutically acceptable carrier. The TEM8 or the fragment thereof may be a recombinant protein or a peptide. The recombinant protein or the peptide may comprise of modified amino acids, unmodified amino acids or both. The TEM8 or the fragment thereof in the composition may have an amino acid sequence shown in SEQ ID NO: 2 or SEQ ID NO: 4.

Alternatively, a nucleotide sequence encoding the immunogenic sequence or the fragment thereof and a nucleotide sequence encoding the TEM8 or the fragment thereof may be inserted in a vector. The vector in such a case may be a plasmid, a viral vector or a bacterial vector. Additionally, the nucleotide sequence encoding the TEM8 or the fragment thereof may comprise at least 60 nucleotides. Furthermore, the nucleotide sequence encoding TEM8 or the fragment thereof in the composition may have a sequence shown in SEQ ID NOS: 1 or 3.

The present invention is further directed to a method of increasing the ability of an immunogenic composition to elicit an immune response in a subject, comprising the step of administering a composition comprising TEM8 or a fragment thereof and a pharmaceutically acceptable carrier to the subject. This method may further comprise administering an immunogenic sequence or a fragment thereof to the subject. The immunogenic sequence or the fragment thereof may be administered prior to, simultaneous with or subsequent to the administration of the composition comprising the TEM8 or the fragment thereof. The immunogenic sequence or the fragment thereof that is administered may be an immunogenic sequence or a fragment thereof of a tumor associated antigen or a pathogen-associated antigen. All other aspects regarding the types of tumor associated antigen, the type of pathogen-associated antigen and the subject in whom the increased immune response are the same as described supra.

The present invention is also directed to a composition comprising an amino acid sequence that is at least 90% homologous to TEM8 or a fragment thereof and a pharmaceutically acceptable carrier. The amino acid sequence of TEM8 or the fragment thereof may have an amino acid sequence shown in SEQ ID NO: 2 or SEQ ID NO: 4. Further, the sequence homologous to TEM8 or the fragment thereof may be a recombinant protein or a peptide. The recombinant protein or the peptide may comprise of modified amino acids, unmodified amino acids or both.

Alternatively, a nucleotide sequence encoding the sequence homologous to TEM8 or the fragment thereof may be inserted in a vector. The vector in such a case may be a plasmid, a viral vector or a bacterial vector. Additionally, the nucleotide sequence encoding the sequence homologous to TEM8 or the fragment thereof may comprise at least 60 nucleotides. Furthermore, the nucleotide sequence encoding TEM8 or the fragment thereof in the composition may have a sequence shown in SEQ ID NOS: 1 or 3.

The present invention is further directed to a method of increasing the ability of an immunogenic composition to elicit an immune response in a subject, comprising the step of administering a composition comprising the sequence that is at least 90% homologous to TEM8 or a fragment thereof and a pharmaceutically acceptable carrier to the subject. This method may further comprise administering an immunogenic sequence or a fragment thereof to the subject. All other aspects regarding the manner of administering the immunogenic sequence or the fragment thereof, types of immunogenic sequence or the fragment thereof and the subject in whom the increased immune response are the same as described supra.

The present invention is also directed to a composition comprising an amino acid sequence that is at least 80% homologous to TEM8 or a fragment thereof and a pharmaceutically acceptable carrier. The amino acid sequence of TEM8 or the fragment thereof may have an amino acid sequence shown in SEQ ID NO: 2 or SEQ ID NO: 4. Further, the sequence homologous to TEM8 or the fragment thereof may be a recombinant protein or a peptide. The recombinant protein or the peptide may comprise of modified amino acids, unmodified amino acids or both.

Alternatively, a nucleotide sequence encoding the sequence homologous to TEM8 or the fragment thereof may be inserted in a vector. The vector in such a case may be a plasmid, a viral vector or a bacterial vector. Additionally, the nucleotide sequence encoding the sequence homologous to TEM8 or the fragment thereof may comprise at least 60 nucleotides. Furthermore, the nucleotide sequence encoding TEM8 or the fragment thereof in the composition may have a sequence shown in SEQ ID NOs: 1 or 3.

The present invention is further directed to a method of increasing the ability of an immunogenic composition to elicit an immune response in a subject, comprising the step of administering a composition comprising the sequence that is at least 80% homologous to TEM8 or a fragment thereof and a pharmaceutically acceptable carrier to the subject. This method may further comprise administering an immunogenic sequence or a fragment thereof to the subject. All other aspects regarding the manner of administering the immunogenic sequence or the fragment thereof, types of immunogenic sequence or the fragment thereof and the subject in whom the increased immune response are the same as described supra.

As used herein, “a sequence that is at least 90% homologous to TEM8 or fragment thereof” may comprise a sequence that is 99%, 98%, 97%, 96%, 95%, 94%, 93%, 92%, 91% or 90% homologous to TEM8 or a fragment thereof. Similarly, as used herein, “a sequence that is at least 80% homologous to TEM8 or fragment thereof” may comprise a sequence that is 99%, 98%, 97%, 96%, 95%, 94%, 93%, 92%, 91%, 90%, 89%, 88%, 87%, 86%, 85%, 84%, 83%, 82%, 81% or 80% homologous to TEM8 or a fragment thereof.

An immunogenic composition comprising a immunogenic sequence or the fragment thereof of a tumor associated antigen or a pathogen-associated antigen may be included along with TEM8 or a fragment thereof or sequences that are at least 90% or at least 80% homologous to TEM8 or a fragment thereof in a same composition or may be in different compositions. Additionally, the immunogenic sequence may comprise one antigen or multiple different antigens. As such, the immunogenic composition may be administered prior to, simultaneous with or subsequent to the administration of TEM8 or the fragment thereof or sequences that are at least 90% or at least 80% homologous to TEM8 or fragment thereof. Either way, the combined effect of such co-administration is to enhance elicitation of immune response by the immunogenic composition.

The composition or compositions described herein, may be administered either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of the composition described herein may comprise conventional non-toxic, physiologically or pharmaceutically acceptable carriers or vehicles suitable for the method of administration and are well known to an individual having ordinary skill in this art.

The compositions described herein may be administered independently one or more times to achieve, maintain or improve upon a therapeutic effect. It is well within the skill of an artisan to determine dosage or whether a suitable dosage of the composition(s) described herein may comprise a single administered dose or multiple administered doses. An appropriate dosage depends on the subject's health, the elicitation of the immune responses and/or treatment of the cancer or pathogen associated disease, the route of administration and the formulation used.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Mice

MMTV-FVB/neuNT mice (strain 233) transgenic for the activated rat neu oncogene (Muller et al., 1988) were purchased from Charles River (Calco, Italy). Four-week old females were routinely screened for the transgene by PCR, as described. FVB/N mice were purchased from Taconic Farms, Inc. (White Plains, N.Y.) and C57BL/6 mice from The Jackson Laboratory (Bar Harbor, Me.). All mice were housed in a pathogen-free vivarium in accordance with institutional guidelines under a protocol reviewed and approved by the Institutional Animal Care and Use Committee of Memorial Sloan-Kettering Cancer Center (MSKCC). All female mice entered the study between 6 and 8 weeks of age.

### Example 2

### Cell Lines and Tissue Culture

The 233-VSGA1 breast tumor cell line was derived from a mouse mammary carcinoma arising in FVB/neuNT mice transgenic for the activated rat neu. This cell line was maintained as described (Nanni et al., 2000)) and was the gift of Dr. Pier-Luigi Lollini (University of Bologna, Bologna, Italy). B16F10 is a spontaneous mouse melanoma tumor of C57BL/6 origin and was from Dr. Isaiah Fidler (MD Anderson Cancer Center, Houston, Tex.). This tumor cell line was maintained as described (Hara et al., 1995)).

### Example 3

### DNA Constructs

The extracellular domain of rat HER2/neu was amplified by polymerase chain reaction from the pCMVneuNT plasmid (Amici et al., 1998), using Platinum® TAq DNA Polymerase High Fidelity (Invitrogen, carlsbad, Calif., USA) and the primers FW 5′-CGAAGCTTACCATGGAGCTGGCGGCCTGG-3′ (SEQ ID NO: 5) and REV 5′-CGGAATTCTTATGTCACCGGGCTGGC-3′. (SEQ ID NO: 6). The HindIII-EcoRI fragment was cloned into pcDNA3.1 (Invitrogen, Carlsbad, Calif.). As a source of cDNA encoding TEM8, total RNA was extracted from a mammary tumor isolated from an FVB/neuNT transgenic mouse. A portion of the extracellular domain of TEM8 (aa 13-357) was amplified by PCR using the following primers: 5′-GGACTCMCGTGGCTGCACTCGTGC-3′ (SEQ ID NO: 7) and 5′-AGAGCAGCGCCAGGGCCAGCAGCAG-3′ (SEQ ID NO: 8).

PCR was performed for 35 cycles at 95° C. for 1 min, 64° C. for 1 min, 72° C. for 2 min, followed by 72° C. for 10 min. PCR products were purified and cloned into pGEM T easy vector (Promega, Madison, Wis.) using standard techniques to create the plasmid pGEMTEM8. For expression of TEM8 in eukaryotic cells and in vivo, a fragment of the TEM8 gene encoding part of the extracellular domain (aa 28-279) was amplified from pGEMTEM8 and sub-cloned into pcDNA3.1 (Invitrogen). The primers were: 5′-GGGGGTACCGCAATGGGCCGCCGCGAGGATGGGGGA-3′ (SEQ ID NO: 9) and 5′-GGTGGAATTCCTAGCACAGCAAATAAGTGTCTTC-3′ (SEQ ID NO: 10). PCR was performed as described above.

The PCR product was then digested with KpnI and EcoRI and cloned into pcDNA3.1 (Invitrogen) to create pcDNATEM8. Large-scale preparation of plasmid was conducted by alkaline lysis using Qiagen Plasmid Giga or Maxi Kits (Qiagen, Venlo, The Netherlands). hTYRP1/hgp75 was previously cloned into the WRG/BEN plasmid (Weber et al., 1998)).

### Example 4

### In Situ Hybridization

The intracellular domain of mouse TEM8 was amplified by PCR and cloned into pGEM T easy vector (Promega) using the following primers: 5′-GAAGACGATGATGGTTTGCCA-3′ (SEQ ID NO: 11) and 5′-GTGGTAGGTGTTGTTCAGGGG-3′ (SEQ ID NO: 12). The cloned products were sequenced and screened against the GENEBANK database (NCBI) to verify mouse TEM8 identity and orientation within the vector. Antisense and sense probes were then generated using T7 and SP6 polymerase (Roche Applied Science, Indianapolis, Ind.) after digestion with either NcoI or SalI, respectively.

Formalin-fixed, paraffin-embedded blocks from 233-VSGA1 breast tumor were sectioned (8 mm thickness), deparaffined, rehydrated and pretreated for in situ hybridization. Hybridization was performed with ³³P-labeled RNA at 65° C. overnight. After removing unbound riboprobes, sections were washed and dehydrated. The slides were dipped in autoradiographic emulsion (NTB-2; Kodak, Rochester, N.Y.) and incubated at 4° C. in a desiccator. Autoradiographic detection was performed after two weeks of exposure. Slides were stained in Gill's hematoxylin, dehydrated, and then mounted with Permount (Sigma, St. Loius, Mo.).

### Example 5

### TEM8 Recombinant Protein

TEM8 recombinant protein was overproduced in bacteria using vectors and procedures supplied in the Gateway Cloning Technology Kit (Invitrogen). Tem8 DNA was amplified from pGEMTEM8 using the primers: attB1bis-5′-GGGGACAAGTTTGTACAAAAAAGCAGGCTTGATGGGCCGCCGCGAG GATGGGGGA-3′ (SEQ ID NO: 13) and attB2bis-5′-GGGGACCACTTTGTACAAGAAAGCTGGGTCGCACAGCAAATAAGTGTCTTC-3′ (SEQ ID NO: 14). The recombinant construct was introduced into E. coli BL21 Star (DE3) competent cells.

Positive transformants were cultured in LB medium until they reached mid-log phase (OD₆₀₀=0.4-0.6), and protein was induced with 0.5 M isopropyl thiogalactoside (IPTG). A HiTrap Chelating HP affinity column (Amersham Biosciences, Piscataway, N.J.) was used to enrich TEM8 protein from cell lysates. A further purification by FPLC using a mono Q anionic exchange column (Amersham) was necessary to obtain a preparation of TEM8 having a single band after Coomassie staining. The purified protein was eluted from mono Q resin using a NaCl gradient. TEM8 protein was eluted at 0.1 M NaCl.

### Example 6

### Western Blot Analysis

Protein samples, either whole lysates or recombinant TEM8 protein, were prepared. For lysates, 10⁷ cells were incubated for 30 min on ice in lysis buffer; 25 mM Tris-HCl pH 7.5, 150 mM NaCl, 1% NP-40, 0.5% Na deoxycholate, 0.1% SDS, 100 mg/ml phenylmethylsulfonyl fluoride (PMSF), 1 mM Na₃VO₄, 2 mg/ml aprotinin, 1 mg/ml leupeptin and 1 mg/ml pepstatin (Sigma). Lysates were cleared by centrifugation at 12,000×g for 10 min at 4° C., and 30 mg were loaded on each lane. Samples were separated on 7.5% Tris-glycine PAGE Precast gels (Cambrex Bio Science, Inc. East Rutherford, N.J.). After electro-transfer, the nitrocellulose membrane (PROTRAN, Schleicher & Schuell, London, UK) was blocked with 5% non-fat dry milk (Bio-Rad Laboratories, Hercules, Calif.) in PBS with 0.05% Tween for 1 h.

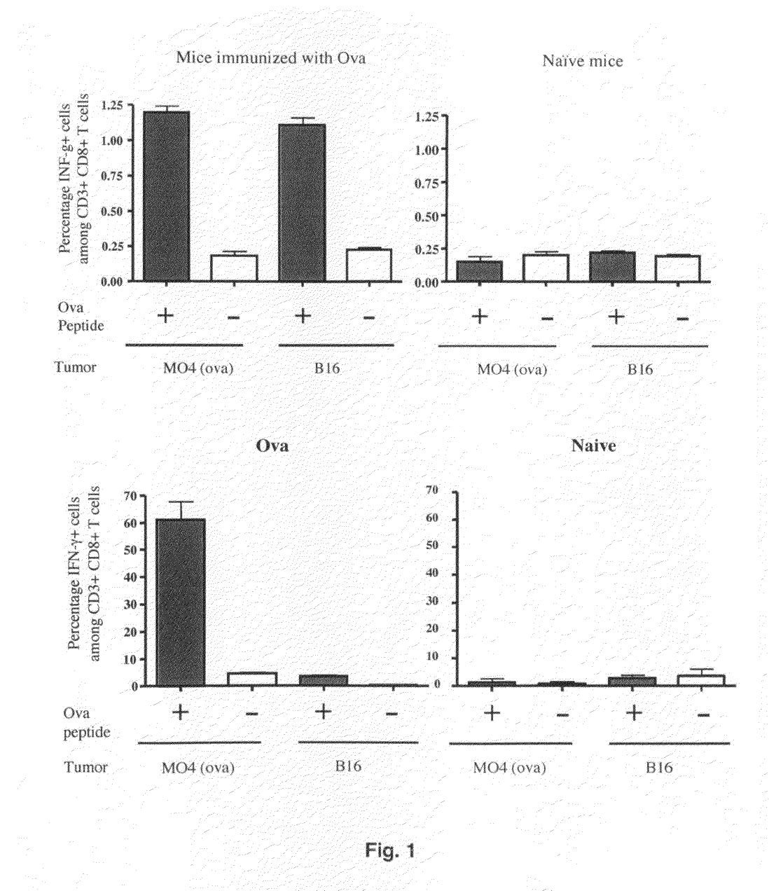

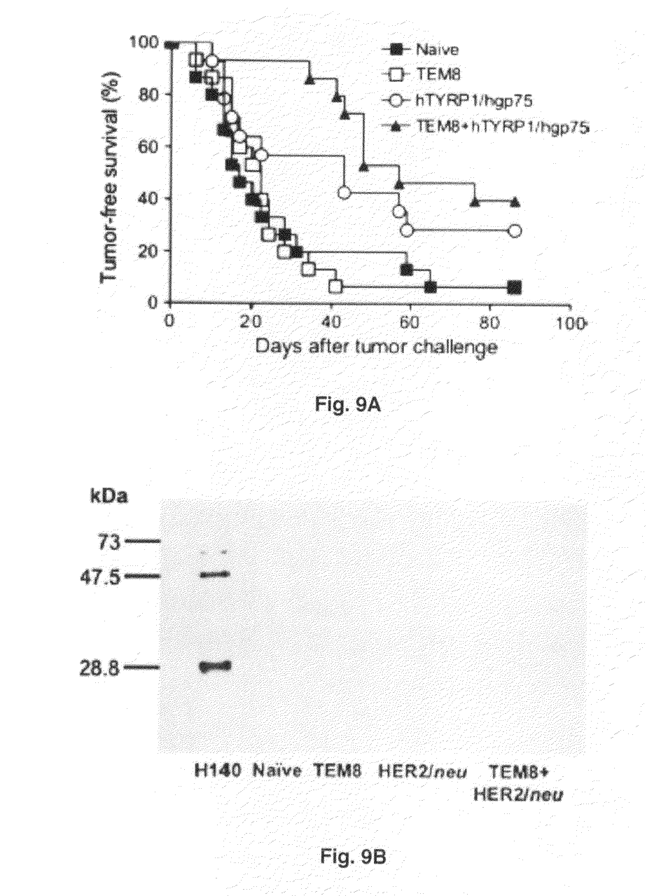

Membranes were probed for 1 h at room temperature with a 1:500 dilution of one of two polyclonal anti-ATR/TEM8 antibodies; N19 (FIG. 1B) or H140 (FIG. 81) or H140 (FIG. 9B) (Santa Cruz Biotechnology, Inc., Santa Cruz, Calif.) in 5% non fat-dry milk PBS/T, or with a 1:100 dilution of pooled mouse sera. Bound antibodies were detected using rabbit anti-goat, goat anti-rabbit or goat anti-mouse IgG-HRP conjugates (Jackson ImmunoResearch Laboratories, Inc., West Grove, Pa.), as appropriate and were visualized using an ECL kit (Amersham).

### Example 7

### DNA and Peptide Immunization

FVB/N and C57BL/6 females were immunized by helium-driven gold particle bombardment, as reported (Ross et al., 1997). Briefly, prior to immunization, the abdominal skin of mice was shaved and depilated with Nair (Church and Dwight, Co., Princeton, N.J.), and the gold particles carrying DNA were injected into the abdominal skin of anesthetized mice using a helium-driven gene gun (PowderMed, Inc., Oxford, UK) at a pressure of 400 psi.

Four injections were delivered, one to each of the abdominal quadrants, for a total of 4 μg plasmid DNA per mouse. Immunization was repeated weekly for 3-5 weeks. Mice treated with a combination of two different DNA plasmids were injected twice per week (days −3 and 0).

For injection of tumor cells in matrigel gel and intracellular cytokine staining (ICCA), FVB/N mice (3/group) were injected once a week for three weeks via particle bombardment with neu or FL-neu DNA or left untreated as negative control (Naïve). Five days after the last vaccination, a HER2/neu+ mammary tumor cell line (233-VSGA1) mixed with matrigel (Becton Dickinson, San Jose, Calif.) were subcutaneously injected into the right flank of each mouse (10⁶ tumor cells in 250 ml of Matrigel/mouse). Matrigel-plugs and spleens were collected 10 days after and ICCA (intracellular cytokine staining) was performed. Effector cells were re-stimulated in vitro with T2Dq target cells (E:T ratio 10:1) pulsed with the immunodominant HER2/neu peptide (RNeu 420). Golgi Stop (BD Pharmingen, San Diego, Calif.) was added to facilitate accumulation of secreted cytokines in the endoplasmic reticulum. Cells were harvested after an over-night re-stimulation period, stained with fluorochrome-conjugated antibodies and analyzed by Flow Cytometry for the production of IFN-γ. For peptide immunization, 50 mg of peptide was mixed with TiterMax (Sigma Aldrich, St. Louis, Mo.) (1:1 ratio) and injected in each footpad (100 mg of peptide/mouse). 7 days after the single peptide injection, mice were sacrificed and poplitelial LNs harvested. Elispot assay was performed as described above.

### Example 8

### Tumor Challenge and In Vivo CD8⁺ T-Cell Depletion

FVB/N and C57BL/6 mice immunized as described above were challenged intradermally with 5×10⁴ 233-VSGA1 or with B16F1025K tumor cells, respectively, in the flank. Tumors were measured by determining two perpendicular diameters three times per week with a caliper and scored positive when they reached 2 mm in any diameter and continued to grow. Mice were sacrificed when the tumor ulcerated or reached 1 cm in any diameter. For in vivo CD8+ depletion, bioreactor culture supernatants from the rat anti-mouse CD8 hybridoma 53-6.7.2 (Guevara-Patino et al., 2006) were injected intraperitoneally into the mice on days −2, +5, +12 and +19 relative to tumor challenge. The first two injections consisted of 250 μg 53-6.7.2 and 500 μg for the third and fourth injections. Each batch of antibody was tested for depletion of CM⁺ T cells prior to its use in immunized mice.

### Example 9

### Preparation of Bone Marrow-Derived Dendritic Cells

Bone marrow was harvested from the femurs of FVB/N nontransgenic mice, washed with PBS, depleted of RBC in ACK lysing buffer (Cambrex Bio Science, Walkersville, Md., USA) and cultured at 1×10⁶/mL in 24-well plates in RPMI 10% FCS, NEAA P/S L-Glu_bme, HEPES and 20 ng/mL recombinant mouse GM-CSF (R&D Systems, Minneapolis, Minn., USA). Cultures were fed on days 2, 4 and 5. Recombinant TEM8 protein (50 mg/1×10⁶ dendritic cells (DC)) was added on day 6. Control DC cultures contained no TEM8 protein. Non-adherent cells (immature DC) were isolated on day 7, replated at 1×10⁶/2 mL in complete media containing GM-CSF and pulsed with an additional 50 mg recombinant TEM8 protein. On day 8, an aliquot of cells was stained with Ab specific for B7.1 and CD11c and 93% of the cells expressed both markers. The remaining cells were used as presenters in an intracellular cytokine secretion assay.

### Example 10

### Intracellular Cytokine Secretion Assay

A single-cell suspension was prepared from the draining lymph nodes of mice immunized with empty vector, TEM8, HER2/neu or HER2/neu+/TEM8. Lymph nodes from naive mice served as additional controls. DC (unpulsed or pulsed with TEM8 protein) were washed with complete RPMI media. Cells were plated (1×10⁶ LN cells₊0.33 10⁵DC in 1 mL final volume) in complete media, for 2 h, and 10 μL 1×BFA (BD Pharmingen, San Diego, Calif., USA) was added. After 16-20 h, cells were stained for surface CD3, CD4, CD8 and intracellular IFNg using the reagents and protocols supplied by the manufacturer (BD Pharmingen). Events (100 000) were acquired on a FACSCAN (BD Biosciences, Franklin Lakes, N.J., USA) and data evaluated using FloJo (FloJo LLC, Ashland, Oreg., USA) 6.3 software.

### Example 11

### ELISpot Assay

TEM8-specific INF-γ production was determined by a standard ELISpot assay (Hawkins et al., 2000). Draining lymph nodes (DLN) were harvested from TEM8-immunized or control C57BL/6 mice 5 days after the last of three DNA injections. DLN were mechanically disrupted in complete RPM1 and CD8+ T cells were positively selected by incubation with magnetic anti-CD8 beads (Miltenyi Biotec Inc, Auburn, Calif., USA). CD8+ T cells (1×10⁵/well) were incubated for 20 h with H2-b target cells (EL-4, 1×10⁴/well) pulsed with one of four TEM8 peptides at 10 mg/mL. These TEM8 peptides, ACYGGFDL (SEQ ID NO: 15), SVLHHWNEI (SEQ ID NO: 16), IYYFVEQL (SEQ ID NO: 17) and NSQGYRTA (SEQ ID NO: 18), were predicted to bind to H2-Kb or H2 Db. As a negative control, target cells were either left unpulsed or pulsed with an irrelevant ovalbumin peptide SIINFEKL (SEQ ID NO: 19). Spot development was performed as described (Herr et al., 1996) and spots were counted using a stereomicroscope and automated computer (Carl Zeiss Inc., Munchen-Hellbergmoos, Germany), counting at 40-fold magnification.

Additionally, inguinal and axillary lymph nodes (LNs) from immunized and control FVB/N mice were harvested five days after the last DNA injection. LNs were mechanically disrupted in complete RPMI. CD8+ T cells were positively selected by incubation with magnetic anti-CD8 beads (Milteny Biotec, Auburn, Calif.). HER2/neu-specific IFN-g production was determined by a standard ELISPOT assay following a 20 hour incubation of CD8+ T cells (1×10⁵/well) with T2Dq target cells (1×10⁴/well) pulsed with RNEU420-429 peptide (10 mg/ml). The RNEU420-429 epitope has been shown to be immunodominant for the H2-Dq haplotype and it maps to the extracellular region of HER2/neu (Ercolini et al., 2003). As a negative control, target cells were left unpulsed.

### Example 12

### Proliferation Assay

The protocol and reagents were supplied with the Cell Proliferation ELISA kit (Roche, Milan, Italy). Spleens and lymph nodes were collected from individual FVB/NT 233 mice immunized with pcDNA3.1, TEM8, HER2/neu and HER2/neu plus TEM8 plasmids. After smashing, splenocytes and lymphocytes were harvested, plated in a 96-well plate (200 000 cells/well) and stimulated with one of two doses of TEM8 recombinant protein (25 or 100 mg/mL) or with Con A (Amersham, Milan, Italy) (1 or 3 mg/mL) for 5 days at 378C at 5% CO₂. Then, 10 mm BrdU (Roche) (final concentration) was added to the cells for 5-6 h. After development, the absorbance was measured at 450 nm using an ELISA plate reader (Lab Systems, M-Medical Srl, Milan, Italy).

### Example 13

### Statistical Analysis

Log Rank analysis was performed to evaluate differences in tumor-free survival based on Kaplan-Meier plots.

### Example 14

### T Cell Responses are Amplified in the Tumor Bed

Injections of TEM8 DNA increased anti-tumor immunity when given in concert with a second, tumor-restricted DNA vaccine and that this effect is in large part dependant on CD8 T cells. These observations were made using two vaccines, HER2/neu in FVB mice, and hgp75 in C57BL/6 mice, that by themselves elicit primarily an antibody response.

A series of experiments to identify CD8 T cells specific for TEM8 in immunized mice were performed. To amplify weak T cell responses, a matrigel assay to expand and detect infiltrating T cells (TILS) in the vicinity of tumors was used. Ovalbumin and HER2/neu were used as model antigens. Mice immunized with a cDNA encoding an ovalbumin peptide were challenged with ova+tumors in matrigel, and after 6 days, the percentage of ovalbumin-specific CD8 T cells in draining lymph nodes and in the matrigel plug containing the tumor was compared (FIG. 1). Controls included naïve mice and mice challenged with tumors that did not express ovalbumin (B16).

Approximately 1% of the CD3+CD8+ T cells in the draining lymph nodes of all mice immunized with ova recognized the ova peptide in an intracellular cytokine secretion assay, regardless of whether the tumor expressed ovalbumin. However, in the tumor bed of ova+tumors, there was a substantial expansion of ova-specific CD8+ T cells, such that 60% of the CD3+ CD8+ T cells in the matrigel plug were ova-specific, compared to 5% for the control ova-tumor. Ova-specific T cells were absent in naïve mice.

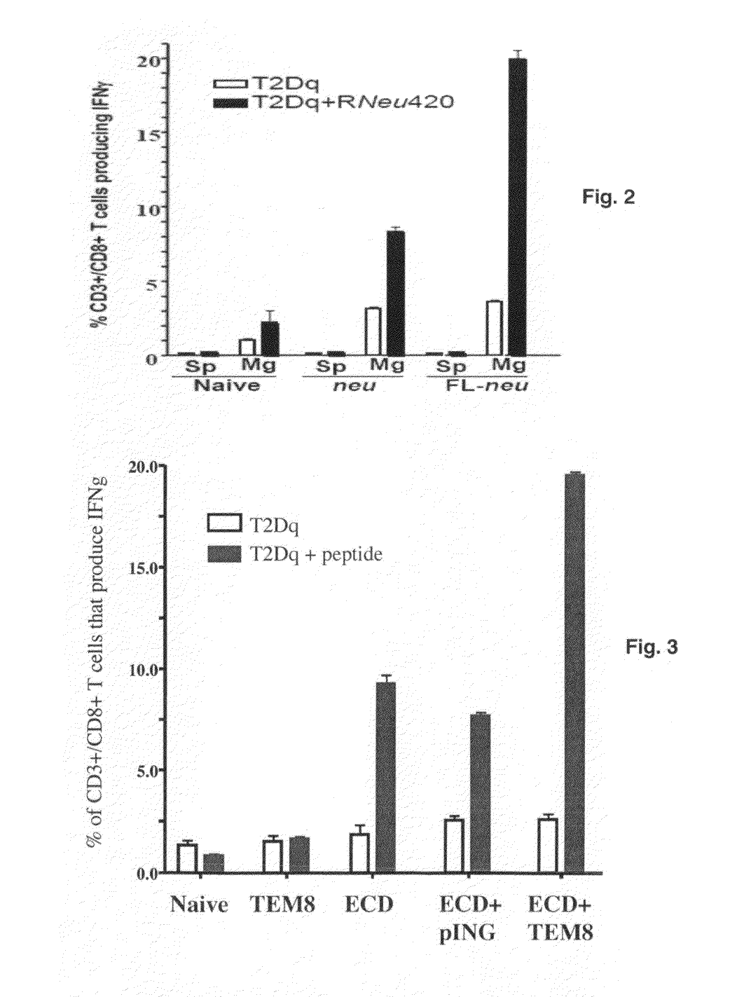

Additionally, mice were also immunized with the rHER2/neu cDNA fused to Flt3-ligand (FL-neu) and challenged with the 233-VSAG1 tumor in matrigel. After 4, 6 or 10 days, the neu-specific response was measured by ICCA using cells isolated from spleen, draining lymph node and matrigel plugs. Starting at 4 days, a neu-specific response was seen (1% of CD3+CD8+ T cells), and this increased to 2% on day 6 and 11% on day 10 (results not shown). At no time did the response in the draining lymph nodes or spleen exceed 0.3%. To confirm the result, mice immunized with HER2/neu or FL-neu were subjected to the same analysis and a clear amplification of neu-specific CD8+ T cells was seen in the tumor bed, at a time when no responding cells are detected in the spleen (FIG. 2).

A computer algorithm was used to predict TEM8 peptides with high avidity to mouse MHC class I Kd and Db, and spleen cells pulsed with four such peptides were used to stimulate T cells from TEM8 immunized mice in ELIspot and intracellular cytokine flow cytometry (ICC) assays. A CD8 T cells specific for TEM8 in immunized mice could not be demonstrated despite using the matrigel assay or a standard IFN-g ELISPOT. Furthermore, antibodies were not seen when sera from immunized mice was used to probe Western blots loaded with recombinant TEM8 protein.

### Example 15

### TEM8 DNA Increases CD8T Cell Response to HER2/Neu and hTRP-1 DNA Vaccines Both at the Tumor Site and in the Draining Lymph Nodes of Tumor Naïve Mice

Faced with the fact that the overall tumor immunity elicited by the combined vaccine was partially dependant on CD8+ T cells, and yet no TEM8-specific CD8+ T cells were observed, the CD8 T cell response to HER2/neu was quantified in mice immunized with HER2/neu alone, TEM8 alone, or the two DNAs in combination, using the matrigel assay. A two-fold increase in the CD8 T cell response to HER2/neu present at the tumor site in tumor bearing mice was observed when both TEM8 and HER2/neu vaccines were given (FIG. 3). Empty vector (pING) had no such adjuvant effect.

This increase in neu-specific T cells could result from improved access to neu positive cells within the tumor caused by inflammation, or a localized immune response to TEM8 protein expressed in endothelial cells or stroma. The possibility that a weak response to TEM8 that was undetectable by ICCA and ELISPOT assays but still sufficient to increase inflammation within the tumor was also considered. Alternatively, TEM8 could alter the immune response in the lymph nodes, by some undefined mechanism.

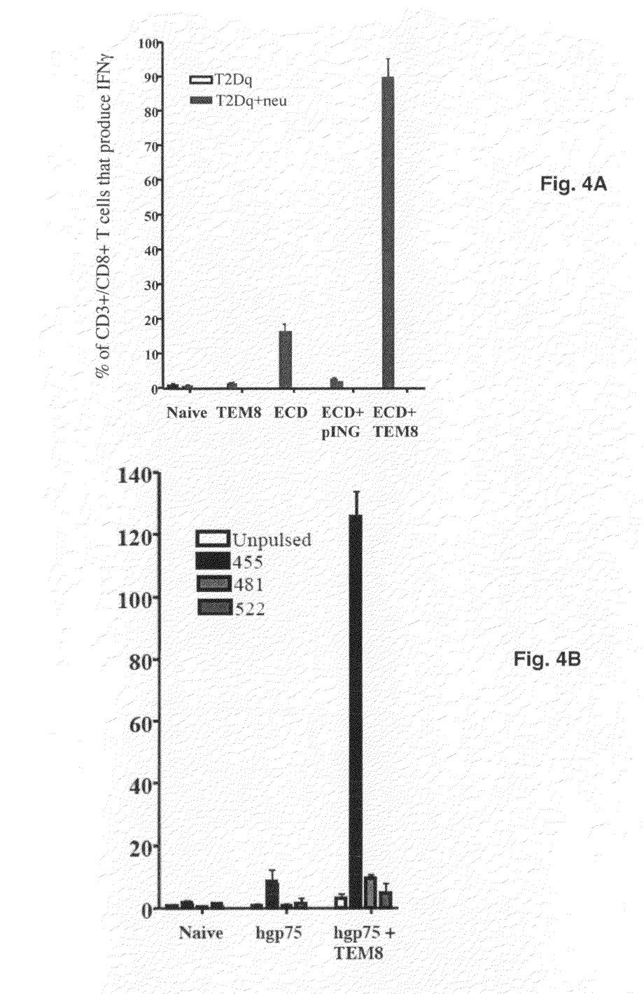

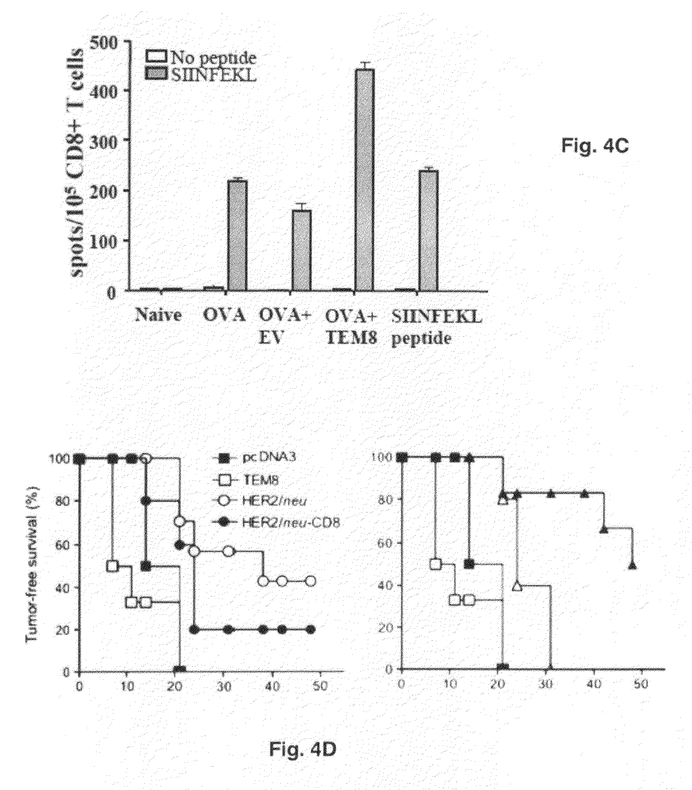

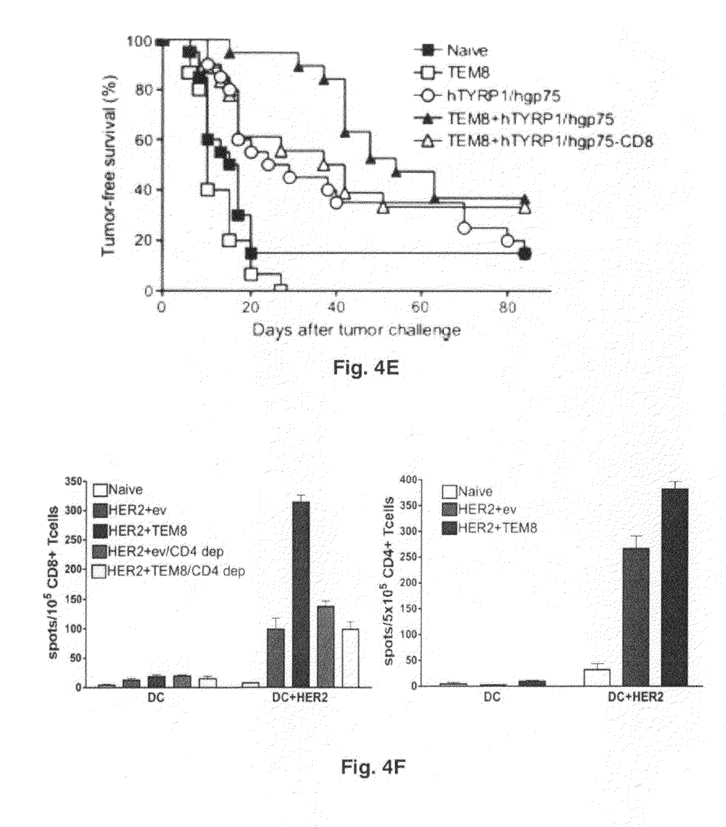

To differentiate between these possibilities, mice were immunized with TEM8, HER2/neu or the two vaccines in combination. In these experiments, tumor was not implanted, but five days after the last vaccine, draining lymph nodes were harvested and assayed for CD8 T cell responses to the immunodominant rneu peptide using the IFN-g ELISPOT (FIG. 4A). TEM8, but not empty vector (pING), increased five-fold the number of reactive cells in the draining LNs of immunized, non-tumor bearing mice. Essentially identical increases in CD8 T cells were also seen when TEM8 was combined with a melanoma differentiation antigen, human tyrosinase-related protein 1 (hgp75) (FIG. 4B). In addition, TEM8 also increased the CD8+ T cell response to a foreign antigen, ovalbumin (FIG. 4C). This shows the general utility of TEM8 as an immune adjuvant for multiple applications in tumor immunology and infectious diseases.

To assess the contribution of CD8 T cells to TEM8-dependent immunity, these cells were depleted in mice just prior to tumor challenge and for 3 weeks afterwards. In vivo depletion of CD8+ T cells was performed by intraperitoneal injections of rat MAb clone 53-6.7.2. Mice immunized with HER2/neu alone were partially protected from tumor challenge (P=0.0004] and this effect was reduced, but not eliminated, by CD8 depletion (P=0.3) relative to HER2/neu (FIG. 4C, left). Mice immunized with the combined vaccine (TEM8+HER2/neu) were also partially protected from tumor challenge (P=0.0009). In this case, tumor protection was completely lost upon T-cell depletion (P=0.02), although there was a delay in tumor growth relative to the control groups (FIG. 4C, right). In the melanoma model, hTYRP1/hgp75 gave partial protection (P=0.1) and the combined vaccine enhanced this protection relative to hTYRP1/hgp75 alone (P=0.04) (FIG. 4D). A combined TEM8+hTYRP1/hgp75 vaccine protected mice from tumor challenge (P=0.0008) and T-cell depletion reduced tumor protection to a level very close to that seen with the hTYRP1/hgp75 vaccine alone (P=0.4).

When syngeneic DCs are transfected with HER2/neu RNA by electroporation, they process and present the entire complement of HER2/neu peptides that are able to bind each MHC I and MHC II molecule. When these cells are used as targets in the ELISPOT, the overall CD8+ and CD4+ T cell responses to all immunogenenic HER2/neu peptides in mice following vaccination in the presence and absence of TEM8 DNA can be quantified. Briefly, the electroporation of DC with mRNA was performed as follows:

Murine DCs were derived from mouse bone marrow (BM). The BM was flushed from tibiae and femorus of naïve mice. Cells were cultured in media supplemented with GM-CSF (32 ng/ml) for 6-7 days. Typically, on day 7 cells were used for electroporation. 4×10⁶ BM-derived DCs were washed twice with PBS and resuspended in 100 ml Solution R (Amaxa, Gaithersburg, Md.); after adding 20 mg of HER2/neu mRNA, cells were pulsed using the AMAXA NUCLEOFECTOR II device (Program U015) and rapidly transferred into culture dishes containing pre-warmed medium. DCs were used as targets in ELISPOT assays 24 hours after transfection. The in vitro transcription of HER2/neu encoding mRNA was performed using the mMESSAGE mMACHINE T7 Ultra kit (Ambion, Austin, Tex.) and following the manufacturer guidelines.

Using this method, a 3 fold increase was observed in overall CD8+ T cells specific for HER2/neu when TEM8 was included in the vaccine, but this increase was completely lost when mice were depleted of CD4+ T cells during immunization. These mice were depleted of CD4 T cells by injecting i.p. an anti-CD4 monoclonal antibody produced by hybridoma clone GK1.5 obtained from MSKCC Monoclonal Antibody facility. The antibody was administered once a week starting 1 day prior to the first DNA immunization and for the duration of the entire immunization phase (250 mg per mouse). Using cells obtained from these same mice, the overall CD4+ T cell response was measured, and there was a modest increase in the number of HER2/neu-specific CD4+ T cells in TEM8 treated mice (FIG. 4E). These data indicate that TEM8 acts in part by increasing CD4+ T cell responses, and this in turn increases CD8+ T cells.

### Example 16

### Mutated TEM8 has No Adjuvant Activity

As a first step to determine whether TEM8 protein has biological activity as an immune adjuvant, wild-type TEM8 was compared to two constructs (Opt-1 and Opt-2) with 9 amino acid changes each. The mutations were introduced into MHC I anchor residues and were designed to increase binding of peptides to H2-Kb and H2-Db. However, these changes are not predicted to increase CD8 T cell immunity in FVB mice.

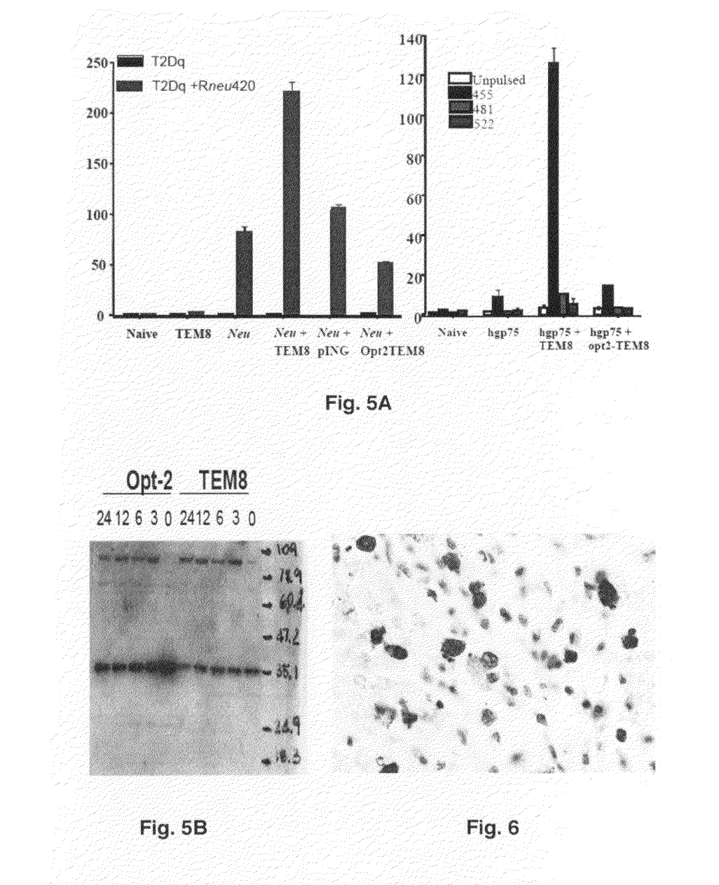

When Opt-2 was used in combination with the model antigens hgp75 (hTRYP-1) or HER2/neu in C57BL/6 or FVB mice respectively, the Opt2 construct had no adjuvant activity (FIG. 5A). To determine the reason for the reduced activity, COS 7 cells were transfected with (FLAG-tagged) Opt-2 or TEM8 DNA. Briefly, FLAG-tagged TEM8 or opt-2 plasmids were obtained cloning TEM8/opto2 sequence downstream (3′) of FLAG in a pcDNA3.1/flag vector (Invitrogen).

COS-7 cells were transfected in 6 well cluster plates with 1 ug DNA using fugene6 (Roche) according to the manufacturer's instruction. Munocomycin was obtained from Sigma, St. Louis, Mo. Cell lysates were separated on a 12% bis-tris acrylamide gel, transferred to PVDF membrane and FLAG-tagged proteins detected using an Anti-FLAG antibody directly conjugated to HRP (Sigma, St. Louis, Mo.) and ECL developing reagents (Pierce). As both TEM8 and Opt2 proteins were stable for 24 hours after transfection into COS 7 cells and subsequent muconomycin A treatment (FIG. 5B), it was concluded that the reduced activity could not be due to reduced protein stability. These data further support the hypothesis that the TEM8 protein acts as an immune adjuvant, and that mutations in Opt-2 inactivate TEM8. Two of the changes in Opt-2 lie in conserved residues in the vWF domain of TEM8 that is shared with other vWF domain proteins.

### Example 17

### TEM8 is Expressed in Monocytes/Macrophages and Dendritic Cells

The data presented above support the possibility that TEM8 protein elaborated by the TEM8 DNA “vaccine” is not the target of a specific immune response but rather acts as an adjuvant. Because prior reports have indicated that macrophages and dendritic cells are targets of anthrax infection, and because these cells are also known to be present in tumors, several methods were used to examine the expression of TEM8 in APCs. Cells expressing TEM8 were shown to be found in the stroma of mouse breast and melanoma tumors. Using IHC, cells expressing TEM8 were visualized in a spontaneous prostate tumor in Pten/p53 conditional null mice (FIG. 6). Western blots have confirmed this observation. It was predicted that at least some of these TEM8+ cells were macrophages.

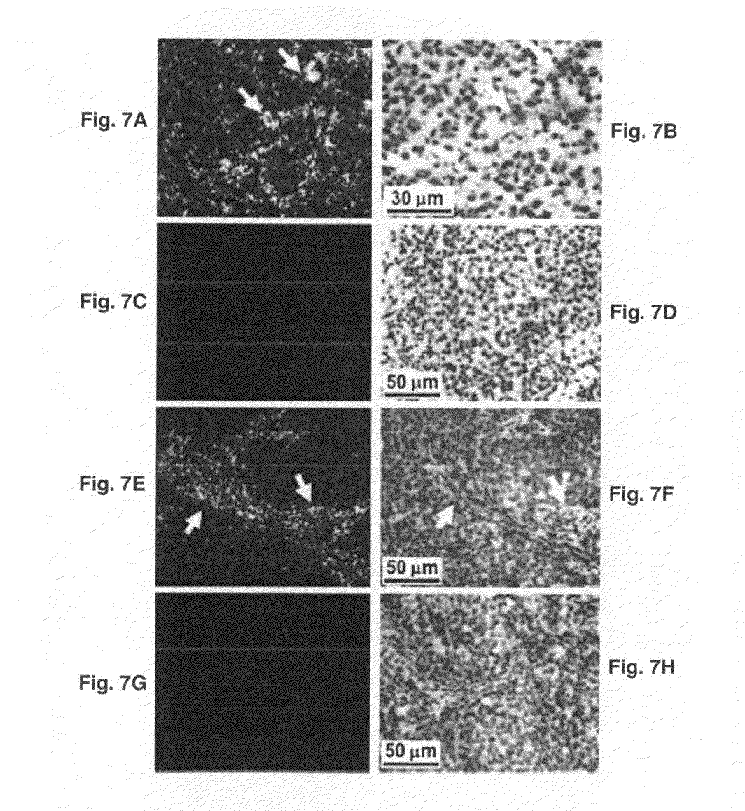

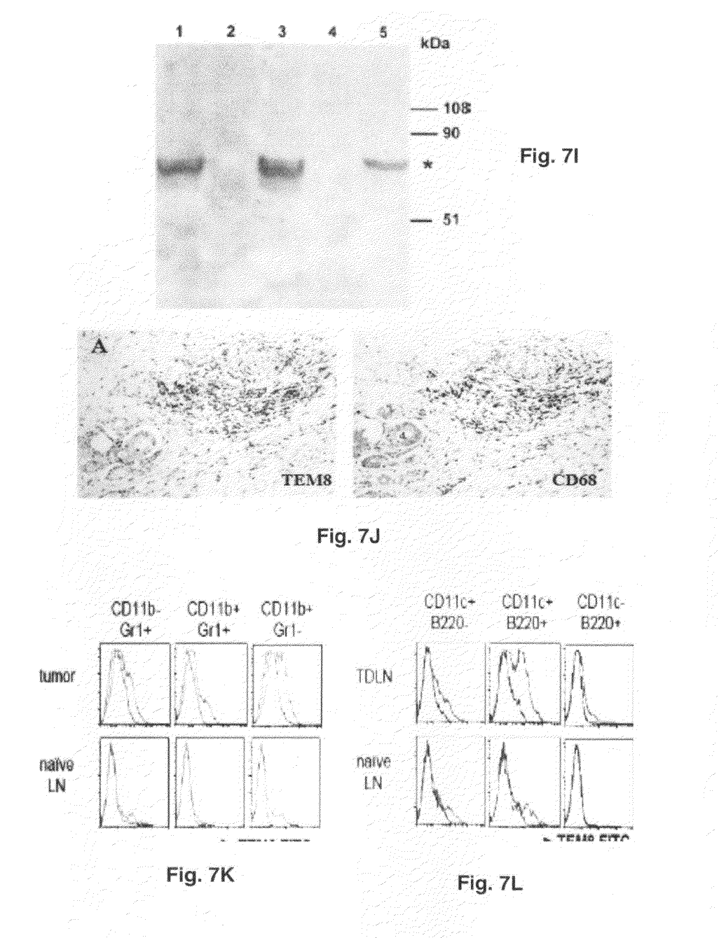

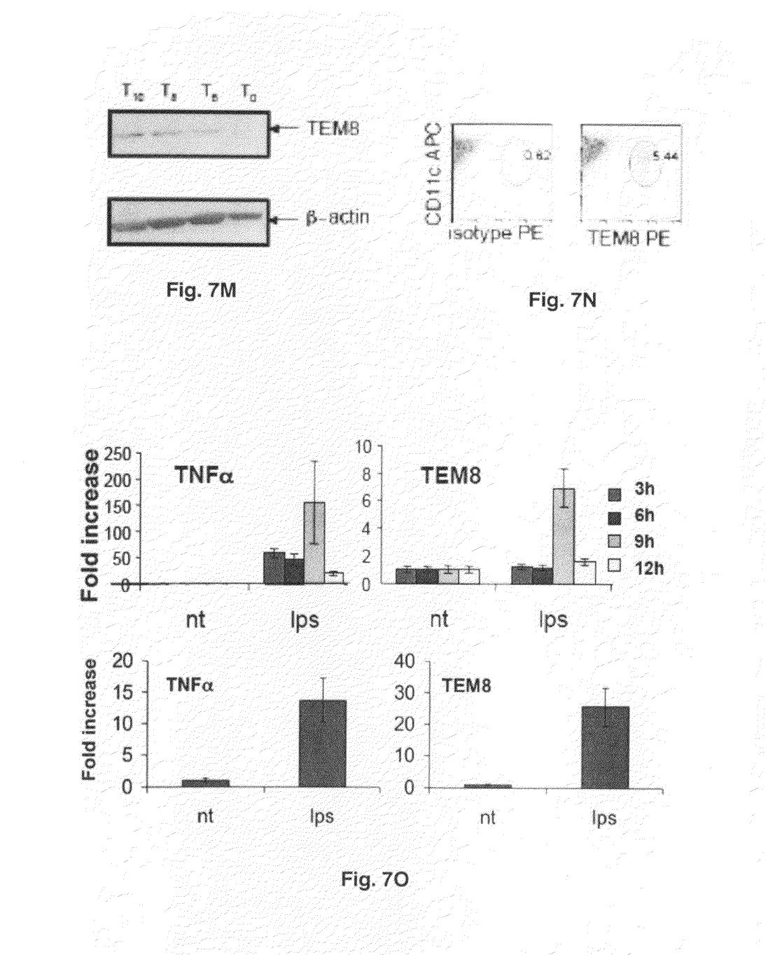

TEM8 was originally described as a transcript whose expression was restricted to tumor endothelial cells, with very limited expression in adult tissues. More recently, TEM8 protein was detected in normal mouse tissues using immunohistochemistry and Western blot analysis (Bonuccelli et al., 2005). The present invention demonstrates TEM8 mRNA expression in the stroma of both breast tumor (233-VSGA1) and melanoma (B16F10) while control ‘sense’ probes showed no hybridization signal (FIGS. 7A-7H). The size of the reactive protein expressed in breast tumors and melanoma, c. 80 kDa, was confirmed in a Western blot (FIG. 7I). This indicated that the predominant protein expressed in these tumors was TEM8 and not ATR or sTEM8. TEM8 was also expressed in normal mouse kidney but not cultured 233-VSGA 1 or B 16F10 tumor cells.

The present invention also demonstrates that TEM8 positive cells within the mouse and human tumor stroma have a morphology typical of the monocyte-macrophage lineage, and co-localize with cells stained with the pan-macrophage marker CD68 (FIG. 7J). Using flow cytometry, TEM8 expressing cells within the tumor are CD11b+ and/or Gr1+, corroborating the hypothesis that these cells are of myeloid origin (FIG. 7K). Interestingly, the same myeloid populations in naïve LN express little to no TEM8, suggesting that the tumor promotes TEM8 expression in myeloid cells or favors the recruitment/proliferation of TEM8+ cells within the tumor stroma.

Surprisingly, although Cd11c+ cells do not express TEM8 in the tumor, the cells expressing TEM8 in the tumor draining lymph-nodes (TDLN), but not in naïve lymph nodes, are mainly CD11c+B220+ plasmacytoid dendritic cells (FIG. 7L). In vitro cultured bone marrow derived DC also express TEM8 (FIGS. 7M, 7N). Briefly, the protocol for the techniques used herein are as follows:

The present invention examined 6 prostate and 9 breast human tumors. Tissues used were formalin fixed and paraffin embedded. For each independent case, 8 mm thickness sections were cut. Slides were deparaffinized in xylene for 30 minutes, rehydrated using graded ethanol concentrations and steamed for 30 minutes at 98° C. in citric acid buffer in a vegetable steamer. Following quenching with hydrogen peroxidase for 5 minutes and biotin blocking using avidin, sections were incubated overnight with a 1:200 dilution of the polyclonal anti-ATR/TEM8 antibody (N19, Santa Cruz Biotechnology) or 1:100 anti-human CD68 (DakoCytomation) antibody in PBS buffer. Detection of antibody binding was achieved using a biotinylated secondary antibody and horseradish peroxidase-conjugated streptavidin (DakoCytomation) and 3′,3′-diamino-benzidine as chromogen. Slides were counterstained with hematoxylin. Appropriate positive and negative control slides were stained in parallel (FIG. 7J).

For the results described in FIG. 7K, LNs and tumors were resected from a day 10 B16-matrigel bearing mouse. Tumors and LN were smashed through a cell strainer. Tumors were additionally centrifuged on a Percoll gradient to enrich a live cell fraction. Tumor cells were stained with CD11bAPC, CD11bAPCCY7, TEM8 (+anti-rabbit PE secondary) and 7AAD. After staining, cells were gated for 7AAD-(live) cells.

For results described in FIG. 7L, LN cells were stained with CD11cAPC, B220Percp and TEM8(+anti-rabbit PE secondary) or CD11b APC, CD11 bAPCCY7 and TEM8(+anti-rabbit PE secondary). Analysis shown in 7C is gated on live cells based on forward and side scatter (FSC-SSC) morphology. For results described in FIG. 7M, DCs were derived from the bone marrow of C57B16 mice femors and cultured for the indicated days with RPMI 10% FBS, Gln, PEN-STREP, beta-mercaptoethanol, 30 ng/ml GM-CSF. A Western blot was performing loading lysates containing bug of total protein on a 10% bis-tris acrylamide gel. For results described in FIG. 7N, flow analysis was gated on DAPI-live cells (CD11cAPC, TEM8+ antirabPE staining is shown). All the antibodies for flow cytometry were purchased from BD, except that the anti-TEM8 (rabbit) was purchased from Affinity bioreagents.

Furthermore, cultured DCs or macrophages significantly increased TEM8 expression after stimulation with the polyclonal activator lipopolysachharide (LPS) (FIG. 70). LPS increased surface expression of the co-stimulation marker CD86 and induced secretion of cytokines IL-6 and IL-12 in DCs. Expression of TEM8 occurred with the same kinetics as TNFα.

Briefly, DC were derived from bone marrow as described supra and macrophages were obtained culturing bone marrows with RPMI 7.5% FBS, Gln, PEN-STREP, 10 ng/ml M-CSF. Day 10 DC and day 5 macrophages were treated with 1 ug/ml LPS (Sigma). Macrophages were harvested 24 h after LPS stimulation, DCs at the indicated time points. RNA was extracted using TRIZOL (invitrogen) according to manufacturer's instruction. 2 ug RNA were retro-transcribed using a High Capacity cDNA archive kit (ABI). 60 ng of cDNA were used as a template for the qPCR reaction.

Commercially available TEM8, TNFα and 18S specific TAQman probes (Applied Biosystems) were used. Reactions were run using a master mix (ABI) and ABI 7500 Thermocycler (ABI). Results indicate the fold increase expression of the indicated RNA, normalized to the relative 18S expression, and to the expression of the non treated sample at the same time point. Taken together these experiments indicated that TEM8 could be a new marker of activated antigen presenting cells (APCs), with particular relevance to tumor immunity and antigen presentation.

### Example 18

### TEM8 DNA Enhances Tumor Immunity when Combined with DNA Vaccines Encoding HER2/neu or TYRP1/gp75

DNA vaccines encoding the extracellular domain of rat HER2/neu inhibit tumor incidence and growth in a transgenic mouse model of spontaneous breast tumorigenesis (Amici et al., 1998, Esserman et al., 1999; Piechocki et al., 2001; Foy et al., 2001; Quaglino et al., 2004) and this effect was enhanced by cytokines (Chen et al., 1998; Pilon et al., 2001; Disis et al., 2003; Croci et al., 2004; Lin et al., 2004; Spadaro et al., 2005). Protection from endogenous breast tumors was also improved when HUVEC was injected in combination with rat HER2/neu DNA (Venanzi et al., 2002).

To develop a molecularly defined vaccine that would be more readily translated into clinical applications, a vascular marker to incorporate into the instant vaccine in lieu of HUVEC cells was determined. TEM8 was chosen as an immune target and the whole cell HUVEC vaccine was substituted with a cDNA encoding a portion of the extracellular domain of TEM8 (aa 28-279). Female transgenic FVB/neuNT mice (strain 233), with mammary expression of the activated rat neu oncogene, were immunized three times bi-weekly using intramuscular injection and then challenged with 233-VSGA 1 tumor cells. This model was chosen for statistical reasons, as the transplanted tumors grow more rapidly and uniformly than endogenous breast tumors. Furthermore, implanted tumors are more readily monitored in living animals. The HER2/neu vaccine as a single agent afforded partial tumor protection (P=0.0002) while TEM8 alone had no activity.

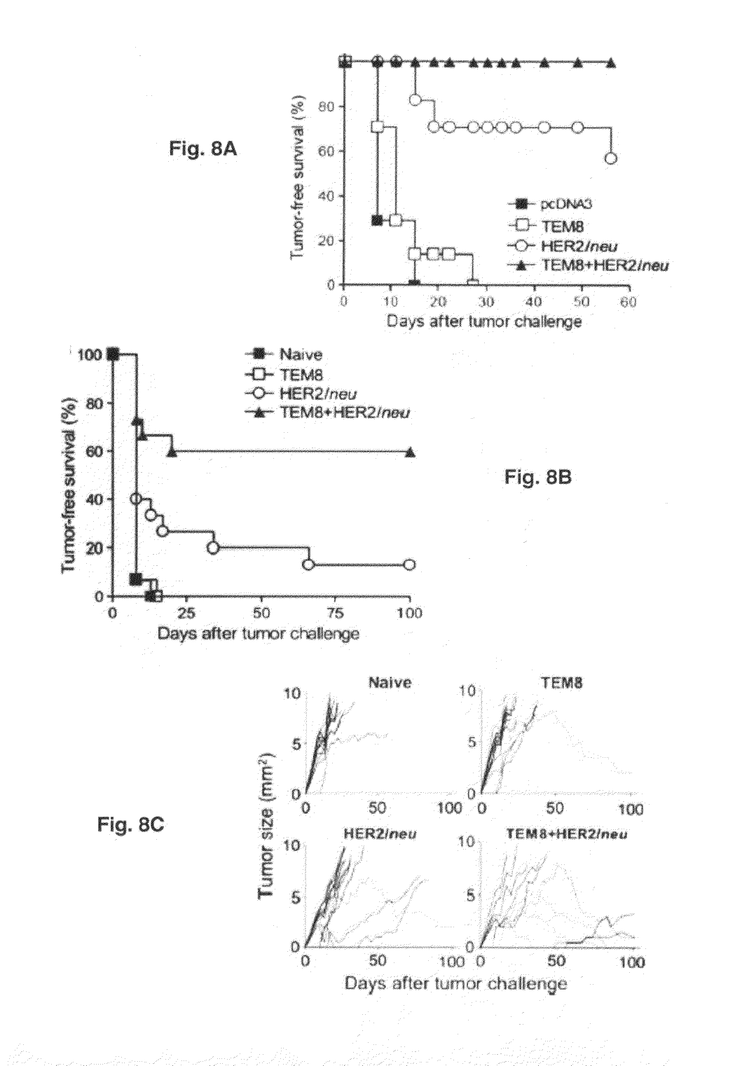

Of the mice immunized with ECD alone, 50% were free of tumors at 56 days after tumor challenge, while co-injection of DNA encoding TEM8 and HER2/neu conferred complete tumor protection in this mouse model; this represented a marked improvement compared with HER2/neu alone (P=0.06) (FIG. 8A). Very similar results were seen in parental, non-transgenic FVB/N mice immunized using a different DNA delivery method, particle bombardment. In place of the co-injection schedule used in intramuscular immunization, a weekly schedule was adopted. Each week, mice (15/group) were injected with TEM8 DNA 3 days prior to HER2/neu DNA injection. Partial protection (15%) from 233-VSGA1 tumor challenge was observed in mice immunized with HER2/neu alone (P=0.01) while TEM8 alone had no activity. Coordinate administration of both HER2/neu and TEM8 vaccines induced long-term (100 days) tumor protection in 60% of the mice (FIG. 8B). The protection afforded by the combined vaccine was significantly better than that seen in control animals (PB/0.0001) and in mice that received TEM8 (P=0.0001) or HER2/neu (P=0.01) alone. Interestingly, it was also observed that four out of 15 FVB/N mice immunized with a combined vaccine (TEM8+HER2/neu) showed initial tumor growth for up to 50 days before they rejected the tumor completely (FIG. 8C).

In order to investigate whether TEM8 DNA injection also increased anti-tumor immunity to other tumor types and in combination with other tumor Ag, TEM8 was combined with a DNA vaccine encoding the melanocyte differentiation Ag, xenogeneic (human) hTYRP1/hgp75. It was shown that xenogeneic melanocyte differentiation Ag induce cross-reactive protective immunity to the corresponding mouse Ag expressed in mouse melanoma B16F10. For example, human TYRP-1/hgp75 DNA provides partial protection from mouse melanoma B16F10, which expresses the closely related mouse TYRP-(80% identity and 90% homology) (Naftzger et al., 1996; Bowne et al., 1999; Perales et al., 2002). Using a weekly schedule as before, mice were immunized for weeks with hTYRP1/hgp75 alone or in combination with TEM8 DNA and challenged with 4 ul/104 B16F10 melanoma cells. The results were similar to those seen with the HER2/neu system: TEM8 DNA alone had no effect on tumor growth, while hTYRP1/hgp75 DNA alone delayed tumor growth and provided partial tumor protection (P=0.1). The combined vaccine further improved tumor-free survival to a level that was significantly different from the one measured in the control group (P=0.002). This was evident 31 days postchallenge, when hTYRP1/hgp75 alone provided 57% tumor-free survival and the combination of hTYRP1/hgp75 and TEM8 provided 93% tumor-free survival (FIG. 9A).

### Example 19

### Immune Response to TEM8 DNA

In an effort to measure immune responses to TEM8, sera from mice immunized with TEM8 DNA alone or in combination with HER2/neu DNA were analyzed for TEM8-specific Ab. The extracellular domain of TEM8 was overexpressed in bacteria and TEM8 recombinant protein was used as the source of Ag in Western blots developed with pools of sera from each group of mice. No Ab reactivity against TEM8 was detected in mice immunized with either the TEM8 DNA vaccine or a combined vaccine (TEM8+HER2/neu) (FIG. 9B).

Several experiments were performed to assess the T-cell response to TEM8 in immunized mice. BM cells were cultured in GM-CSF and IL-4 for 7 days. The resulting immature DC were pulsed with recombinant TEM8 protein for 2 days and used as targets in an ex-vivo IFNg intracellular cytokine assay with cells isolated from the DLN of mice immunized with TEM8 DNA. No specific recognition was observed. Peptides were synthesized from the primary TEM8 sequence that had a high predicted binding to H2 Kb or H2 Db. After ex-vivo stimulation of CD8+ T cells from mice immunized with TEM8 DNA, there was no IFN-g release in the presence of potential cognate peptide.

Furthermore, a proliferation assay in which splenocytes from immunized mice were stimulated with recombinant TEM8 protein also gave no evidence of specific recognition. Because TEM8 is expressed in some normal tissues, mice injected five times at weekly intervals with TEM8 DNA were subjected to a complete necropsy to identify any areas of autoimmune pathology. With the exception of minor plasma cell hyperplasia and mild multifocal sinus histocytosis in the spleen and hyalinosis in the gall bladder that is commonly seen in C57BL/6 mice, there were no significant pathologic lesions reported in the 25 organs examined. The heart, liver, lung, kidney, spleen uterus and skin all appeared normal.

### Example 20

### TEM8 injection increases DC Migration from Skin to Draining Lymph Nodes

The present invention examined the mechanism by which TEM8 enhanced the immune response discussed supra. In order to do so, migration of DC after injection of TEM8 DNA or emty vector was compared. Briefly, mice were injected with empty vector or TEM8 DNA using the GeneGun. One, 3, 5 or 7 days later mice (shaved on the belly) were treated with 10 ul of a skin-sensitizer solution (1:1 acetone:dibutylphtalate) containing 3.3% FITC and 5% DMSO.

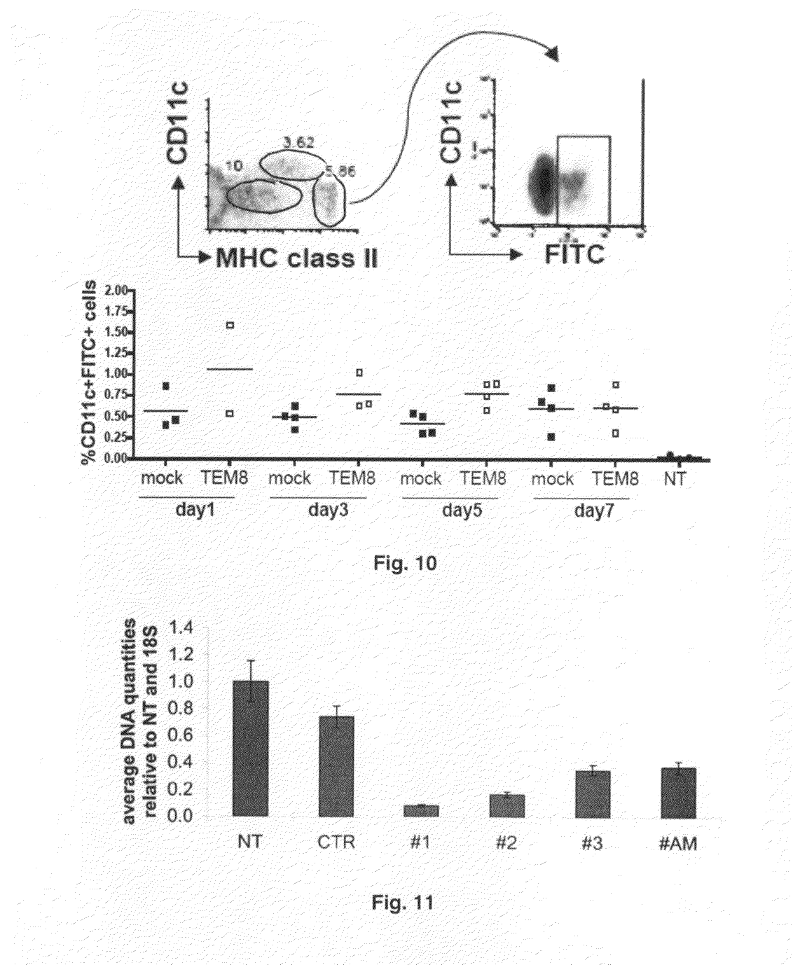

Twenty-four hours after FITC painting, draining LNs were collected and processed for flow cytometry as follows: First, tissue was incubated for 1 hour in PBS with 1 mg/ml collagenase at 37° C. Then, LNs were smashed through a cell strainer and washed with PBS. 2 million cells were stained with CD11cAPC, MHCII pe, CD3APCCY7, CD19TexasRED. At day 3 and 5 following TEM8 administration, there was a significant increase in the percentage of DCs migrating from the skin to the draining lymph nodes as compared to empty vector treated mice (FIG. 10).

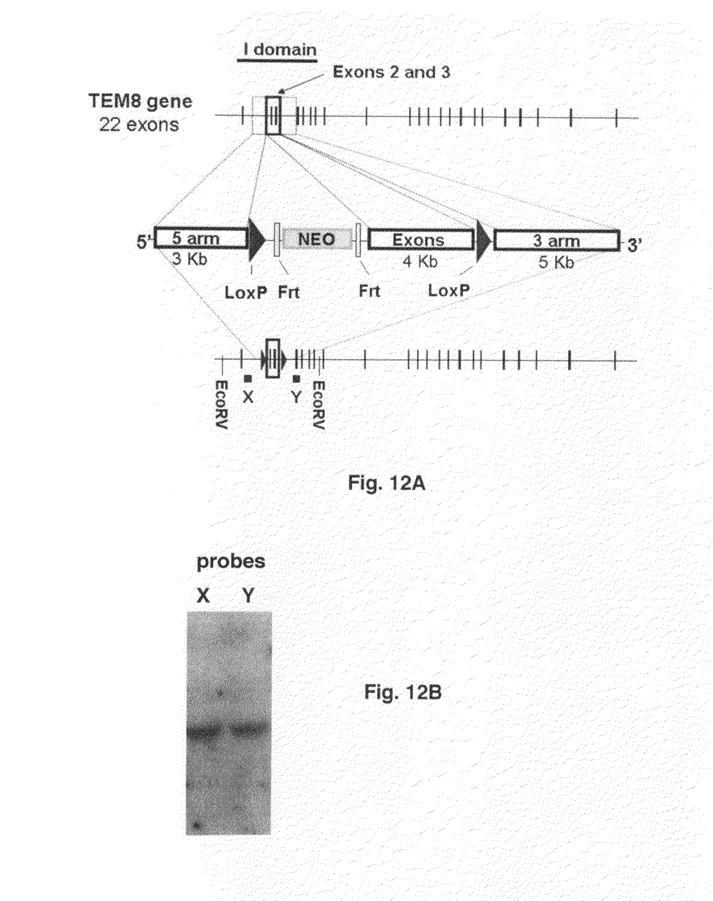

To further explore the role of TEM8 in antigen presentation and T cell activation, several siRNAs were designed and tested for their ability to suppress TEM8 RNA after transient transfection. Briefly, NIH-3T3 cells were seeded in 12 well plates and transfected with 25 nM concentration of the indicated siRNA using lipofectamine-2000 (Invitrogen) according to the manufacturer's instruction. Oligos #1, #2, #3 and CTR were purchased from Sigma-proligo, #am was purchased from ambion. 24 hrs later cells were trypsinized, washed and RNA was extracted using Trizol (Invitrogen) as described above. cDNA was prepared as above and analyzed by qPCR. Of the three siRNAs that were tested, two decreased TEM8 expression at least 5-fold (FIG. 11). These will be used to knock down TEM8 in APCs in vitro, and test APC motility, and antigen presentation.

To study the role of TEM8 in APC maturation and function in vivo, a series of TEM8 conditional null mice are created. The targeting vector was created as follows: Three segments of the TEM8 genome were amplified by PCR. These are a “5′ arm” of 3 Kb flanking exons 2 and 3, a 4 kb fragment including exons 2 and 3, and a 5 Kb “3′ arm” downstream of these exons. Each fragment was cloned and sequenced, and then introduced into the targeting vector as shown. Probes X and Y labeled with ³²P using the Amersham Readyprime II labeling kit were used to detect the TEM8 gene in a Southern blot.

Thus, a targeting vector in which loxP sites were introduced flanking exons 2 and 3 of the TEM8 locus was created. Upon expression of the Cre recombinase, exons 2 and 3 in the TEM8 locus will be excised, disrupting the I-domain of the protein as well as the reading frame (FIGS. 12A-12B). This vector was transfected into ES cells, and colonies were screened for accurate homologous recombination events. After introduction of the selected ES clone in embryos, mice are crossed to other strains in which CRE recombinase is expressed in DCs, macrophages, or other defined cell types.

This effect on DC migration could be explained by three possible mechanisms 1) direct transfection of dermal/epidermal DCs with TEM8 increases DC migratory capability 2) transfection of keratinocytes, which are in close contact to DCs in the skin, indirectly promotes DC migration from the skin 3) TEM8 administration to the skin activates systemic inflammation and lymphatic function, and in turn LNs ‘attract’ more skin DCs. To distinguish between these possibilities, DC migration after TEM8 administration was measured from the site of the skin directly treated by gene gun (lower belly), from a site that is not directly addressed by gene gun but is drained by the same LNs (lower back), or from a site that is not directly addressed by gene gun and that is drained from an unrelated LN (ear). DCs coming from the area of the skin directly transfected with TEM8 have increased migratory properties.

The phenotype of the FITC+DCs was characterized in the LN, focusing on three well established DC populations: LN-resident DCs (CD11c^{hi}, MHCII^{int}), skin-derived DCs (Cd11c^{lo}, MHC II^{hi}) and pDCs (CD11c^{lo}, MHCII^{lo}. TEM8 did not increase the migration of skin-derived DCs (which include Langerhans cells and dermal-DCs) and lymph node resident DCs; however, a significant effect on the mobility of pDCs was observed.

DC allostimulatory activity after TEM8 injection was analyzed using DCs isolated from the inguinal lymph nodes using magnetic beads conjugated to anti-CD11c antibody. Purified DCs were then co-incubated for 5 days with different ratios of CD4 T cells purified from the spleen of allogeneic Balb/c mice and T cell proliferation assessed by ³H-Thy incorporation. TEM8-treated DCs have an increased capability to induce proliferation of allogeneic CD4+ T cells. These findings support the thesis that TEM8 increases tumor immunity through increased activation of DCs. TEM8 administration locally increases DC migration as well as DC stimulatory activity in the draining LN. Moreover TEM8 injection has a very specific effect on the pDC subset.

A TAQman probe from exon 3 was used to quantify TEM8 mRNA expression by qPCR. TEM8 mRNA is detectable, although at very low level, in all the mouse tissues analyzed (lung heart muscle, intestine, spleen liver thymus, kidney, lymph node and brain, with higher expression in the spleen as compared to all the other organs. To address whether TEM8 was ubiquitously expressed in all the cells of the spleen or only in some specific cell subsets, splenocytes of a naïve WT mouse were FACS sorted using the cell surface markers CD45, CD3 and CD19 and analyzed TEM8 expression in different cell populations of the spleen. TEM8 was not expressed in CD45⁻ cells of the spleen, which include endothelial cells. TEM8 is also not expressed in CD45+CD3+ T lymphocytes and CD45⁺CD19⁺ B cells, instead TEM8 expression is restricted to a population of CD45+CD3⁻CD19⁺ splenocytes. These cells include phagocytes of myeloid origin (dendritic cells and macrophages), granulocytes, NK cells and bone marrow precursors of cells of the immune system. These data support that TEM8 was expressed in inflammatory myeloid cells of the tumor infiltrate. Bone marrow derived myeloid cells (such as macrophages, dendritic cells and their precursors) are a major component of the tumor microenvironment and modulate tumor progression by providing pro-tumoral factors and by orchestrating tumor-specific adaptive immune responses. Thus, TEM8 has a role in immunity both in normal conditions and in cancer.

### TEM8 Injection Increases Inflammatory Cytokine Expression in the Draining Lymph Node

In order to measure changes in cytokine levels after TEM8 injection, RNA was isolated from the skin and dLNs of individual mice 6 hours, 1, 3 and 5 days after injection of TEM8 DNA or empty vector. Quantitative RT-PCR was used to compare the levels of inflammatory or maturation cytokines IL-6, IL-1a, IFNa, IFNg and TNFa; Treg anti-inflammatory cytokines TGFb and IL-10; TH1 cytokines IL-12p35, IL-12p40 and IFNg; TH2 cytokines IL-4 and IL-13 and the TH17 cytokine IL-17. For most cytokines, the changes were minimal, but for IL-6 and IL-1a a 2-3 fold increase in RNA was observed 6 to 12 hours post injection, returning to baseline by day 3. IL-17 increased 3-4 fold in that same time frame, and also returned to baseline by day 3. Changes in protein levels of 64 cytokines and chemokines including those tested by qRT-PCR were measured in the skin and draining lymph nodes 24 hours post injection using semi-quantitative immunoblotting. MIP-2, TCA-3 and TNF-a were increased slightly in the skin after TEM8 injection. TEM8 injection increased the CD8+ and CD4+ T cell responses to HER2/neu.

Many of these studies were performed in the FVB strain, where T cell recognition of the immunodominant RNEU₄₂₀ peptide was measured. In order to ensure the phenomena would extend to another mouse strain, BALB/C mice (H2-d) were immunized with HER2/neu DNA alone, or with TEM8 DNA, and the CD8+ T cell response was measured by IFN-g ELISPOT using spleen cells pulsed with a panel of peptides which were predicted to bind H2d. In every case where a measurable response was seen to a given neu peptide, TEM8 increased that response.

The following references were cited herein:
Amici et al., 1998, Cancer Immunol Immunothera 47: 183-90.
Carson-Walter et al., 2001, Cancer Res. 61:6649-55.
Bonuccelli et al., 2005, Am J Physiol Cell Physiol 288: C1402-1410.
Bowne et al., 1999, J Exp Med 190: 1717-22.
Chen et al., 1998, Cancer Res, 58: 1965-71.
Croci et al., 2004, Cancer Res, 64: 8428-34.
Disis et al., 2003, Immunology, 207: 179-86.
Essermann et al., 1999, Cancer Immunol Immunother, 47: 337-42.
Ercolini et al., 2003, J Immunol 170(8):4273-80.
Felicetti et al., 2007, Cytotherapy 9(1): 23-34.
Foy et al., 2001, Vaccine, 19: 2598-606.
Guevara-Patino et al. 2006, J Clin Invest 116(5):1382-90.
Hara et al., 1995, J Exp Med 182: 1609-14.
Hawkins et al., 2000, Surgery 128: 273-280.
Herr et al., 1996, J Immunol Methods, 191: 131-42.
Lin et al., 2001, Mol Ther 10: 290-301.
Muller et al., 1988, Cell 54:105
Nanni et al, 2000, Int J Cancer 87: 186-94.
Naftzger et al., 1996, Proc Natl Acad Sci USA 93: 14809-14814.
Perales et al., 2002, Semin Cancer Biol 12: 63-71.
Piechocki et al., 2001, J Immunol, 167: 3367-74.
Pilon et al., 2001, J Immunol 167: 3201-6.
Quaglino et al., 2004, Cancer Res, 64: 2858-64.
Ross et al., 1997, Clin Cancer Res, 3: 2191-6.
Spadaro et al., 2005, Clin Cancer Res, 11: 1941-52.
Venanzi et al., 2002, Proc Am Assoc Can Res 43: 90, abstract 448.
Weber et al., 1998, J Clin Invest, 102: 1258-64.

1. A composition, comprising:
an immunogenic sequence or a fragment thereof;
a TEM8 or a fragment thereof;
a pharmaceutically acceptable carrier;
or a combination thereof.

2. The composition of claim 1, wherein the immunogenic sequence or the fragment thereof or the TEM8 or the fragment thereof is a recombinant protein or a peptide.

3. The composition of claim 2, wherein the recombinant protein or the peptide comprises modified amino acids, unmodified amino acids or both.

4. The composition of claim 1, wherein the TEM8 or the fragment thereof has an amino acid sequence shown in SEQ ID NO: 2 or SEQ ID NO: 4.

5. The composition of claim 1, wherein a nucleotide sequence encoding said immunogenic sequence or the fragment thereof and a nucleotide sequence encoding said TEM8 or the fragment thereof is inserted in a vector.

6. The composition of claim 5, wherein the vector is a plasmid, a viral vector or a bacterial vector.

7. The composition of claim 4, wherein the nucleotide sequence encoding said TEM8 or said fragment thereof comprises at least 60 nucleotides.

8. The composition of claim 4, wherein the nucleotide sequence encoding said TEM8 or the fragment thereof has a sequence shown in SEQ ID NO: 1 or SEQ ID NO: 3.

9. The composition of claim 1, wherein the immunogenic sequence or the fragment thereof is an immunogenic sequence or a fragment thereof of a tumor associated antigen or a pathogen-associated antigen.

10. The composition of claim 9, wherein the tumor associated antigen is Her2/NEU, gp75/TYRP-1, PSMA, TRYP-2, NY-ESO-1, MAGE-1, MAGE-3, CEA, PSA, tyrosinase, mutant p53, mutant p21, mutant cdk4, mutant L9, BCR-ABL or E6/E7 of HPV16.

11. The composition of claim 9, wherein the pathogen associated antigen is a bacterial lipopolysaccharide, anthrax lethal factor, anthrax edema factor, HIV gp120 or malaria antigens, wherein the malaria antigens are CSP, TRAP/SSP2, LSA1, MSP1 and AMA-1.

12. A method of eliciting an enhanced immune response in a subject, comprising the step of:
administering an immunologically effective amount of the composition of claim 1 to the subject.

13. The method of claim 12, wherein the TEM8 or the fragment thereof in said composition increases the ability of the immunogenic sequence or the fragment thereof in the composition to elicit an antibody response, a CD4 T cell response, a CD8 T cell response or a combination thereof against an antigen associated with a cancer or against an antigen associated with a pathogen in the subject.

14. The method of claim 12, wherein said antigen associated with a cancer is Her2/NEU, gp75/TYRP-1, PSMA, TRYP-2, NY-ESO-1, MAGE-1, MAGE-3, CEA, PSA, tyrosinase, mutant p53, mutant p21, mutant cdk4, mutant L9, BCR-ABL or E6/E7 of HPV16.

15. The method of claim 12, wherein said antigen associated with a pathogen is bacterial lipolysaccharide, anthrax lethal factor, anthrax edema factor, HIV pg120 or malaria antigens, wherein the malaria antigens are CSP, TRAP/SSP2, LSA1, MSP1 and AMA-1.

16. The method of claim 12, wherein said subject has breast cancer, prostate cancer, melanoma, colon cancer, chronic myeloid leukemia or cervical cancer or has been exposed or suspected of being exposed to malaria, anthrax, HIV or biological weapons.

17. The composition of claim 1, wherein said fragment of TEM8 has an amino acid sequence that is at least 90% homologous to an amino acid sequence of TEM8.

18-27. (canceled)

28. A method of eliciting an enhanced immune response in a subject, comprising the step of:
administering an immunologically effective amount of the composition of claim 17 to the subject.

29-32. (canceled)

33. The composition of claim 1, wherein said fragment of TEM8 has an amino acid sequence that is at least 80% homologous to an amino acid sequence of TEM8.

34-43. (canceled)

44. A method of eliciting an enhanced immune response in a subject, comprising the step of:
administering an immunologically effective amount of the composition of claim 33 to the subject.

45-48. (canceled)

49. A composition, comprising:
a TEM8 or a fragment thereof and a pharmaceutically acceptable carrier.

50. The composition of claim 49, wherein the TEM8 or the fragment thereof is a recombinant protein or a peptide.

51. The composition of claim 50, wherein the recombinant protein or the peptide comprises modified amino acids, unmodified amino acids or both.

52. The composition of claim 49, wherein the TEM8 or the fragment thereof has an amino acid sequence shown in SEQ ID NO: 2 or SEQ ID NO: 4.

53. The composition of claim 49, wherein a nucleotide sequence encoding said TEM8 or the fragment thereof is inserted in a vector.

54. The composition of claim 53, wherein the vector is a plasmid, a viral vector or a bacterial vector.

55. The composition of claim 53, wherein the nucleotide sequence encoding said TEM8 or said fragment thereof comprises at least 60 nucleotides.

56. The composition of claim 53, wherein the nucleotide sequence encoding said TEM8 or the fragment thereof has a sequence of SEQ ID NO: 1 or SEQ ID NO: 3.

57. A method of increasing the ability of an immunogenic composition to elicit an immune response in a subject, comprising the step of:
administering an immunologically effective amount of the composition of claim 49 to the subject.

58. The method of claim 57, further comprising:
administering an immunogenic sequence or a fragment thereof to the subject.

59. The method of claim 58, wherein the immunogenic sequence or the fragment thereof is administered prior to, simultaneous with or subsequent to the administration of the composition comprising the TEM8 or the fragment thereof.

60. The method of claim 58, wherein the immunogenic sequence or the fragment thereof is an immunogenic sequence or a fragment thereof of a tumor associated antigen or a pathogen associated antigen.

61. The method of claim 60, wherein said tumor associated antigen is Her2/NEU, gp75/TYRP-1, PSMA, TRYP-2, NY-ESO-1, MAGE-1, MAGE-3, CEA, PSA, tyrosinase, mutant p53, mutant p21, mutant cdk4, mutant L9, BCR-ABL or E6/E7 of HPV16.

62. The method of claim 60, wherein said pathogen associated antigen is bacterial lipolysaccharide, anthrax lethal factor, anthrax edema factor, HIV gp120 or malaria antigens, wherein the malaria antigens are CSP, TRAP/SSP2, LSA1, MSP1 and AMA-1.

63. The method of claim 57, wherein the immune response comprises an antibody response, a CD4 T cell response, a CD8 T cell response or a combination thereof.

64. The method of claim 57, wherein said subject has breast cancer, prostate cancer, melanoma, colon cancer, chronic myeloid leukemia or cervical cancer or has been exposed or suspected of being exposed to malaria, anthrax, HIV or biological weapons.

65. The composition of claim 49,
wherein said fragment of TEM8 has an amino acid sequence that is at least 90% homologous to an amino acid sequence of TEM8.

66-72. (canceled)

73. A method of increasing the ability of an immunogenic composition to elicit an immune response in a subject, comprising the step of:
administering an immunologically effective amount of the composition of claim 65 to the subject.

74-80. (canceled)

81. The composition of claim 49,
wherein said fragment of TEM8 has an amino acid sequence that is at least 80% homologous to an amino acid sequence of TEM8.

82.-88. (canceled)

89. A method of increasing the ability of an immunogenic composition to elicit an immune response in a subject, comprising the step of:
administering an immunologically effective amount of the composition of claim 81 to the subject.

90-96. (canceled)
